SEQUENCE LISTING (28 sequences)

SEQ ID NO: 1 - DNA, 20 bp, Artificial sequence
  Single strand DNA oligonucleotide
aatttgccaagctcctgaag

SEQ ID NO: 2 - DNA, 20 bp, Artificial sequence
  Single strand DNA oligonucleotide
cgtttggctgaataccttcc

SEQ ID NO: 3 - DNA, 20 bp, Artificial sequence
  Single strand DNA oligonucleotide
tcccaaacaccagtttcctc

SEQ ID NO: 4 - DNA, 20 bp, Artificial sequence
  Single strand DNA oligonucleotide
tgtgtctgcattgctccttc

SEQ ID NO: 5 - DNA, 21 bp, Artificial sequence
  Single strand DNA oligonucleotide
ttgtttcgtaacacagcattg

SEQ ID NO: 6 - DNA, 21 bp, Artificial sequence
  Single strand DNA oligonucleotide
ggcactagtatctttggctga

SEQ ID NO: 7 - DNA, 21 bp, Artificial sequence
  Single strand DNA oligonucleotide
cctacaacaagctgaaaacca

SEQ ID NO: 8 - DNA, 21 bp, Artificial sequence
  Single strand DNA oligonucleotide
ggatagcacagcacaagatca

SEQ ID NO: 9 - DNA, 21 bp, Artificial sequence
  Single strand DNA oligonucleotide
agcagggtctgagaatgaaat

SEQ ID NO: 10 - DNA, 21 bp, Artificial sequence
  Single strand DNA oligonucleotide
gccattgattttgtggttatg

SEQ ID NO: 11 - DNA, 23 bp, Artificial sequence
  Single strand DNA oligonucleotide
ttctacaagatgatcaaggaagg

SEQ ID NO: 12 - DNA, 20 bp, Artificial sequence
  Single strand DNA oligonucleotide
agaattgatccgcacagaat

SEQ ID NO: 13 - DNA, 20 bp, Artificial sequence
  Single strand DNA oligonucleotide
acgaggtccgctacgtaaat

SEQ ID NO: 14 - DNA, 24 bp, Artificial sequence
  Single strand DNA oligonucleotide
ggctctccagacattttacatatt

SEQ ID NO: 15 - DNA, 21 bp, Artificial sequence
  Single strand DNA oligonucleotide
aaagaagtcgcagacgaagga

SEQ ID NO: 16 - DNA, 21 bp, Artificial sequence
  Single strand DNA oligonucleotide
tattggatggtggcattttca

SEQ ID NO: 17 - DNA, 20 bp, Artificial sequence
  Single strand DNA oligonucleotide
accacataggcgctttcctt

SEQ ID NO: 18 - DNA, 21 bp, Artificial sequence
  Single strand DNA oligonucleotide
tatcctagaggtggcggattt

SEQ ID NO: 19 - DNA, 21 bp, Artificial sequence
  Single strand DNA oligonucleotide
gaatggtatcacccagggaat

SEQ ID NO: 20 - DNA, 20 bp, Artificial sequence
  Single strand DNA oligonucleotide
tggaaacagagatgctggtg

SEQ ID NO: 21 - DNA, 20 bp, Artificial sequence
  Single strand DNA oligonucleotide
cctctgttccgtgttggatt

SEQ ID NO: 22 - DNA, 16 bp, Artificial sequence
  Single strand DNA oligonucleotide
aaggctgcagtggaca

SEQ ID NO: 23 - DNA, 21 bp, Artificial sequence
  Single strand DNA oligonucleotide
ctgcatcatcatctccgtctt

SEQ ID NO: 24 - DNA, 21 bp, Artificial sequence
  Single strand DNA oligonucleotide
ccatttatcagcaacctgcat

SEQ ID NO: 25 - DNA, 21 bp, Artificial sequence
  Single strand DNA oligonucleotide
gtgcaatttccactggttgat

SEQ ID NO: 26 - DNA, 21 bp, Artificial sequence
  Single strand DNA oligonucleotide
ggaacggattcagataaggaa

SEQ ID NO: 27 - DNA, 21 bp, Artificial sequence
  Single strand DNA oligonucleotide
ttcacaacatctgagacacca

SEQ ID NO: 28 - DNA, 20 bp, Artificial sequence
  Single strand DNA oligonucleotide
ccggctggggatatatagaa

(19) United States
(12) Patent Application Publication
(10) Pub. No.: US 2020/0199527 A1
(43) Pub. Date: Jun. 25, 2020

(54) METHODS OF MATURATION OF HUMAN SPERMATOGONIUM

(71) Applicant: B.G. Negev Technologies & Applications Ltd., at Ben-Gurion University, Beer-Sheva (IL)

(72) Inventor: Mahmoud HULEIHEL, Beer-Sheva (IL)

(73) Assignee: B.G. Negev Technologies & Applications Ltd., at Ben-Gurion University, Beer-Sheva (IL)

(21) Appl. No.: 16/638,483

(22) PCT Filed: Aug. 17, 2018
(86) PCT No.: PCT/IB2018/056215

§ 371 Date: Feb. 12, 2020

Related U.S. Application Data
(60) Provisional application No. 62/546,658, filed on Aug. 17, 2017.

(51) Int. Cl. C12N 5/076

(52) U.S. Cl. CPC C12N 5/061; C12N 2513/00; A61K 45/06; C12N 2501/11; C12N 2501/31; C12N 2501/115

(57) ABSTRACT

Provided are methods of in vitro maturation of human spermatogonium, comprising culturing the spermatogonium in a three-dimensional methylcellose culture system (MCS) under conditions capable of differentiating said human spermatogonium into an elongated spermatid, thereby in vitro maturing the human spermatogonium. Also provided is an in vitro matured sperm obtainable according to the method of the invention and methods of treating subjects in need of mature sperm cells.

### FIELD AND BACKGROUND OF THE INVENTION

The present invention, in some embodiments thereof, relates to methods of in vitro maturation of human spermatogonium.

Childhood cancer is estimated to affect 0.1% of prepubertal boys up to 15 years of age [1]. Among those, acute lymphoblastic leukemia (ALL) affects around 26% of the patients. About 80% will survive the disease due to the progress in anti-cancer treatments [2-4]. These anti-cancer treatments are mostly gonadotoxic (chemotherapy and/or radiotherapy), which in some cases, are a combination of different types of chemotherapeutic agents or a combination with radiotherapy and may lead to impairment of fertility and even to permanent azoospermia. This depends on the type of dose and combination of anti-cancer treatment agents [5]. These agents may affect both spermatogenic and testicular somatic cells. However, some adolescent patients (16-68%) will become azoospermic following chemo-/radiotherapy [5]. Since prepubertal males do not produce spermatozoa, sperm cell cryopreservation for fertility preservation is unfeasible in this age group. The only suggested possibilities for their fertility preservation are testicular tissue or cell cryopreservation before aggressive anti-cancer treatments, for future use as autotransplantation, or in vitro maturation of their spermatogonial stem cells (SSCs) to sperm (6-9). The feasibility and safety of fertility preservation in prepubertal cancer patient boys via cryopreservation of testicular biopsies has been reported [23-25]. Also, it was shown that testicular growth of the biopsied testis was similar to the non-biopsied contralateral testis until one year after surgery [26]. The limitation of using testicular tissue or cells for autotransplantation is the possibility of presence of residual cancer cells which may restore the disease. For instance, microinjection of rat T-cell leukemia (around 20 leukemia cells) mixed with germ cells into rat testis resulted in a cancer relapse [27]. Today, there is no safe methodology to isolate cancer cells from testicular tissue of cancer patients [10-12].

On the other hand, intratesticular transplantation of mouse spermatogonial stem cells that were grown in vitro into busulfan-treated mice did not affect cancer incidence or the long-term survival rate compared to non-transplanted busulfan-treated mice [28].

Additionally, significant limitation of this approach is the scarce number of SSCs present in the testicle relatively to other germ cell population. In adult mouse testes, this was estimated to be 0.03% [13], the biopsies obtained are very small, and spermatogonial cells comprised about 3% of the cell population of testicular biopsies from prepubertal boys [29]. Considering the small volume of the biopsy, it can be realized that the number of SSCs would be extremely low.

Recently, the capacity of induction propagation of human SSCs from adult and prepubertal boys was demonstrated (14,15). However, none of the published in vitro methodology could induce differentiation of human SSCs to meiotic and postmeiotic stages.

In animal model, Sato et al., using organ culture of testis from immature mouse demonstrated the capacity of induction SSCs to meiotic and postmeiotic stages including the generation of fertile sperm in vitro (16). In this system, the microenvironment niches of the SSCs and the cellular interactions in the seminiferous tubule and in the interstitial compartments remained intact, and thus enabled proliferation and differentiation of the SSCs.

Using isolated spermatogonial cells from immature mouse Abu Elhija M, et al., 2012 could induce their proliferation and differentiation to meiotic and postmeiotic stages including the generation of sperm-like cells in vitro using three-dimension (3D) in vitro soft-agar culture system (SACS) (17-19). Recently, Huleihel M, et al., 2015 could induce proliferation and differentiation of spermatogonial cells from prepubertal monkeys to meiotic and postmeiotic cells in 3D methylcellulose culture system MCS (20).

### SUMMARY OF THE INVENTION

According to an aspect of some embodiments of the present invention there is provided a method of in vitro maturation of human spermatogonium, comprising culturing the spermatogonium in a three-dimensional methylcellulose culture system (MCS) under conditions capable of differentiating the human spermatogonium into an elongated spermatid, thereby in vitro maturing the human spermatogonium.

According to an aspect of some embodiments of the present invention there is provided an in vitro matured sperm obtainable according to the method of some embodiments of the invention.

According to an aspect of some embodiments of the present invention there is provided cell obtainable according to the method of some embodiments of the invention, wherein said cell is characterized by at least the expression of CREM (cAMP responsive element modulator).

According to an aspect of some embodiments of the present invention there is provided a method of treating a subject in need of mature sperm cells, comprising:

(a) obtaining a spermatogonium from the subject, and; (b) subjecting the spermatogonium to an in vitro maturation according to the method of some embodiments of the invention, thereby generating mature sperm cells of the subject, and treating the subject.

According to some embodiments of the invention, the conditions comprise culturing the human spermatogonium in a culture medium which comprises an effective concentration of at least one growth factor selected from the group consisting of TNF-alpha, Glial cell line-derived neurotrophic factor (GDNF), leukemia inhibitory factor (LIF) and basic fibroblast growth factor (bFGF).

According to some embodiments of the invention, the conditions comprise culturing the human spermatogonium in a culture medium which comprises an effective concentration of at least one growth factor selected from the group consisting of Glial cell line-derived neurotrophic factor (GDNF), leukemia inhibitory factor (LIF), basic fibroblast growth factor (bFGF) and epidermal growth factor (EGF)

According to some embodiments of the invention, the culture medium further comprises TNFalpha (TNFα).

According to some embodiments of the invention, the culture medium further comprises at least one agent selected from the group consisting of: testosterone, follicle stimulating hormone (FSH) and retinoic acid.

According to some embodiments of the invention, the culture medium comprises serum replacement.

According to some embodiments of the invention, the culture medium comprises STEM PRO® (Thermo Fisher Scientific) supplement.

According to some embodiments of the invention, the method further comprising culturing the human spermatogonium in the presence of at least one hormone selected from the group consisting of: Follicle-Stimulating Hormone (FSH) and testosterone.

According to some embodiments of the invention, the culture medium further comprises at least one hormone selected from the group consisting of: Follicle-Stimulating Hormone (FSH) and testosterone.

According to some embodiments of the invention, the conditions comprise culturing the human spermatogonium in a culture medium which comprises an effective concentration of at least one growth factor selected from the group consisting of GDNF, LIF, and bFGF.

According to some embodiments of the invention, the conditions comprise culturing the human spermatogonium in a culture medium which comprises an effective concentration of at least one growth factor selected from the group consisting of GDNF, LIF, and EGF.

According to some embodiments of the invention, the conditions comprise culturing the human spermatogonium in a culture medium which comprises an effective concentration of at least one growth factor selected from the group consisting of GDNF, bFGF, and EGF.

According to some embodiments of the invention, the conditions comprise culturing the human spermatogonium in a culture medium which comprises an effective concentration of at least one growth factor selected from the group consisting of LIF, bFGF, and EGF.

According to some embodiments of the invention, the culturing in the presence of the at least one hormone is performed following about one month of culturing in the presence of the at least one growth factor.

According to some embodiments of the invention, the at least one hormone is added to the culture medium which comprises the at least one growth factor.

According to some embodiments of the invention, the method further comprises identifying a meiotic cell, a post meiotic cell and/or a mature sperm cell following said culturing in vitro.

According to some embodiments of the invention, the method further comprises identifying a cell expressing CREM (cAMP responsive element modulator), following said culturing in vitro.

According to some embodiments of the invention, the human spermatogonium is comprised in a testicular biopsy of the subject.

According to some embodiments of the invention, the testicular biopsy is obtained from a prepubertal male subject.

According to some embodiments of the invention, the testicular biopsy is obtained from a non-obstructive azoospermic patient.

According to some embodiments of the invention, the subject is a prepubertal male subject.

According to some embodiments of the invention, the prepubertal male subject is in need of aggressive chemotherapy and/or aggressive radiotherapy.

According to some embodiments of the invention, the male subject is in need of aggressive chemotherapy and/or aggressive radiotherapy.

According to some embodiments of the invention, the prepubertal male subject is diagnosed with cancer.

According to some embodiments of the invention, the prepubertal male subject is diagnosed with autoimmune disease.

According to some embodiments of the invention, the prepubertal male subject is diagnosed with thalassemia.

According to some embodiments of the invention, the prepubertal male subject is in need of bone marrow transplantation.

According to some embodiments of the invention, the cancer comprises a hematological cancer.

According to some embodiments of the invention, the cancer comprises a solid tumor.

According to some embodiments of the invention, the subject is a non-obstructive azoospermic patient.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C depict a non-limiting representation of the study design according to some embodiments of the invention. FIG. 1A—A scheme of design of the study, which includes all the steps of using the biopsies of the patients. First step is to divide the biopsy (according to its size; for each part at least 3 mm³ tissue was used) to be used for tissue culture (first priority), histology and RNA extraction. FIG. 1B—The isolated cells to be used for in vitro culture are divided to cells for before culture (“Be”; as a control of the type of cells were present in the biopsy before culture) or cells for culture. The cells used for culture are followed up for their growth and development in MCS. Every 10 days to weeks a new media was added with growth factors. Testicular biopsies were enzymatically treated (as described hereinbelow) and the isolated cells were cultured in methylcellulose culture system (MCS) which provided 3D in vitro to mimic the 3D of the seminiferous tubules in vivo. The MCS was composed of methylcellulose (e.g., about 42%) and the other percentages composed of a base medium (e.g., StemPro) and various growth factors (GFs) as described hereinbelow, e.g., GDNF, LIF, FGF and EGF. FIG. 1C—The developed spermatogenic cells were examined according to specific markers known to be expressed on cells of premeiotic (stages “As” through “B”), meiotic (spermatocytes; “SPC” and round spermatids; “RS”) or postmeiotic (“RS” and elongated spermatids; “ES”) stages.

FIGS. 2A-N depict histology and immunofluorescence staining of testicular sections from pre-pubertal cancer patient boys. FIGS. 2A-C—Histological sections of testicular biopsy from pre-pubertal cancer patient boys (FIG. 2A—7-years old FIG. 2B—6-years old; and FIG. 2C—6-years old) stained by H&E (hematoxylin and eosin). The histological sections show seminiferous tubule (ST) with the spermatogonial cells A_{dark} (Ad) and Aₚₐₗₑ (Ap), peritubular cells (PTC), spermatocytes (SPC) and interstitial tissues (IST) between the seminiferous tubules that composed of Leydig cells and blood vessels (BV). FIGS. 2D-K—Testicular biopsies from pre-pubertal cancer patient boys (n=3) were examined for the presence of pre-meiotic cells by immunofluorescence staining using specific primary antibodies for the examined pre-meiotic markers; VASA (FIG. 2H), c-KIT (FIG. 2I), GFR-a1 (FIG. 2G), CD-9 (FIG. 2J), α-6-integrin (FIG. 2E), OCT-4 (FIG. 2D) and PLZF (FIG. 2F), compared to the negative control (NC; FIG. 2K) without primary antibodies. Blue color-cells nuclei stained with DAPI [(4′,6-diamidino-2-phenylindole)], red color—the specific marker staining. Magnifications: FIGS. 2A-C: X20; FIGS. 2D-K: X40; light microscope magnification (100 μm scale). FIG. 2L—Magnification of an area from the tissue presented in FIG. 2C, showing the presence of peritubular cells (PTC; white arrows) surrounding the seminiferous tubules and clusters of Leydig cells (LC; black arrows) in the interstitial compartment. FIG. 2M—Immunofluorescence staining of a section from patient No. 2 showing Sertoli cells in the seminiferous tubules (red staining for vimentin). FIG. 2N—A double immunofluorescence staining (orange color) for Sertoli cells (red; vimentin) and GDNF (green) was detected. Magnification of FIGS. 2L-N—X40 light microscope magnification (100 μm scale).

FIGS. 3A-D depict morphology of developed colonies and sperm in vitro from isolated cells obtained from testicular biopsies of pre-pubertal cancer male patients. Germ cells enzymatically isolated from testicular biopsies of pre-pubertal cancer male patients developed after 5-15 weeks of culture to small (about 10/well), medium (about 4-5/well) and large (about 1-3/well) colonies in MCS (methylcellulose culture system). FIG. 3A—Single (s)/pair (p)/aline (al) cells; FIG. 3B—small colonies (up to 30 cells); FIG. 3C—medium colony (up to 100 cells); and FIG. 3D—large colony (>150 cells).

FIGS. 3E-L show double immunofluorescence staining of the premeiotic cells [SALL4 (FIG. 3F) and PLZF (FIG. 3J); green color] with Ki (a marker of proliferation; (FIGS. 3E and 31), red color), DAPI (blue color) stains the nucleus and merged images (FIGS. 3H and 3L) showing the double staining. FIG. 3H is a merge of FIGS. 3E, 3F and 3G. FIG. 3L is a merge of FIGS. 3I, 3J, and 3K. Negative control shows no staining of the markers used.

FIGS. 3M-3T—Morphology and immunofluorescence staining of meiotic and post-meiotic cells developed in MCS. After 15 weeks in culture, cells were harvested and stained immediately with MitoTracker to identify cells with sperm-like morphology [green color, FIGS. 3Q, 3R and 3S). FIG. 3T—shows spermatozoa from a biopsy obtained from azoospermic patient with sperm (used as positive control for MitoTracker staining and shows the morphology of mature sperm). After staining with MitoTracker, the slides were fixed and reused for IF staining with anti-acrosin antibodies (primary antibodies) and Cy3 as secondary antibodies (FIGS. 3M, 3N and 3O). FIG. 3P—is a negative control for acrosin staining. Fluorescence and inverted microscope X40. Blue color—cells nuclei stained with DAPI; red color—the specific marker staining. T—tail; H—head; N— Neck.

FIGS. 4A-N—immunofluorescence staining (FIGS. 4A-M) and RNA expression (FIG. 4N) of testicular cells before and/or after culture in MCS. Cells enzymatically isolated from testicular biopsies of prepubertal cancer patients boys before and/or after culture in MCS were examined by IF staining (FIGS. 4A-M) or PCR analysis (FIG. 4N) for different pre-meiotic [VASA (FIG. 4B), SALL-4 (FIG. 4D), PLZF (FIG. 4C), OCT-4 (FIG. 4A), c-KIT (FIG. 4H), α-6-INTEGRIN (FIG. 4G), CD-9 (FIG. 4F) and GFRc-1 (FIG. 4E); from cells before or after culture], meiotic [CREM-1 (FIG. 4I), LDH (FIG. 4J) and BOULE (FIG. 4K); from cells before or after culture] and post-meiotic [ACROSIN (FIG. 4L); from cells after culture] markers using specific primary antibodies or primers respectively for each examined marker. Negative control [“NC”; (FIG. 4M)] for IF staining was performed without primary antibodies. The sizes of the PCR products were 90 bp-373 bp. (100 μm scale). “Lad”=ladder (black arrow indicates 500 bp). Negative control for PCR analyses was ultra pure water instead of the cDNA.

These results show the proliferation, colony formation and development of spermatogenic cells in vitro from spermatogonial cells isolated from testicular biopsies of prepubertal cancer patients.

FIGS. 5A-N depict immunofluorescence staining of pre-meiotic markers in human testicular biopsies from patients with hypospermatogenesis and Sertoli Cell Only Syndrome (SCOS). Testicular biopsy from patients having hypospermatogenesis (FIGS. 5A, 5B, 5C, 5G, 5H, 5I and 5J) or SCOS (FIGS. 5D, 5E, 5F, 5K, 5L, 5M, and 5N) were examined for the presence of pre-meiotic cells by immunofluorescence staining using specific primary antibodies for the examined pre-meiotic markers; VASA (FIGS. 5A and 5D), c-KIT (FIGS. 5B and 5E), GFRα1 (FIGS. 5C and 5F), CD-9 (FIGS. 5G and 5K), α-6-integrin (FIGS. 5H and 5L), OCT-4 (FIGS. 5I and 5M) and PLZF (FIGS. 5J and 5N). Blue color—cells nuclei stained with DAPI, red color—the specific marker staining.

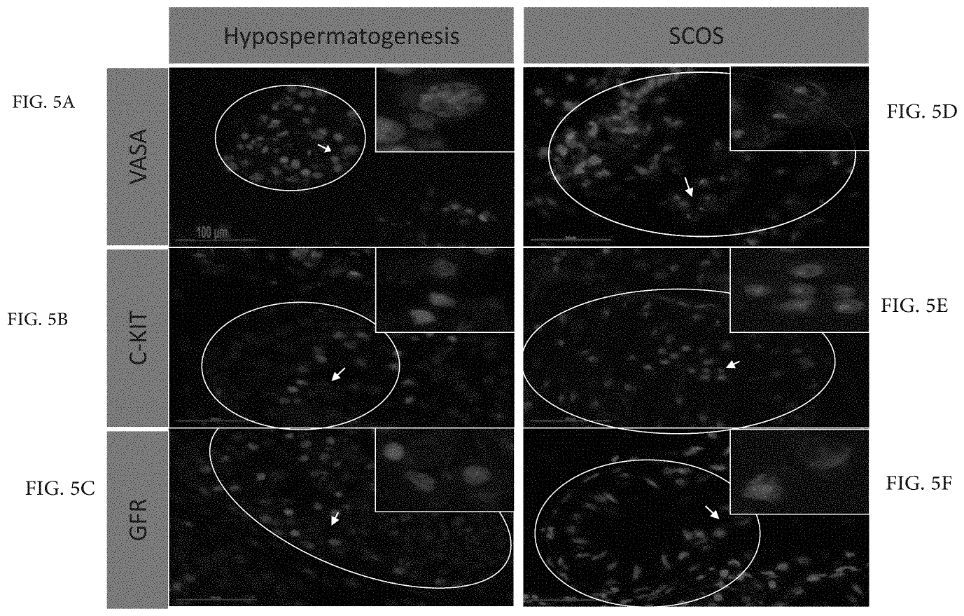

FIGS. 6A-C depict immunofluorescence staining and RNA expression of spermatogenesis markers in cells isolated from biopsies of azoospermic patients. Cells enzymatically isolated from testicular biopsies of azoospermic patients were examined by IF staining (FIGS. 6A-B) or PCR analysis (FIG. 6C) for different pre-meiotic (FIG. 6A; VASA, SALL-4, PLZF, OCT-4, c-KIT, α-6-INTEGRIN, CD-9 and GFRα-1), meiotic (FIG. 6B; CREM-1, LDH and BOULE) and post-meiotic (FIG. 6B; PROTAMINE and ACROSIN) markers using specific primary antibodies or primers respectively for each examined marker. Negative control (FIG. 6B, marked as “NC”) for IF staining was performed without primary antibodies. Blue color—cells nuclei stained with DAPI, red color—the specific marker staining. FIG. 6C—Shows RNA expression of the various markers. RT-PCR analysis with primers specific to the indicated markers. Negative control for PCR analyses was ultra pure water instead of the cDNA. The sizes of the PCR products were 100 bp-200 bp.

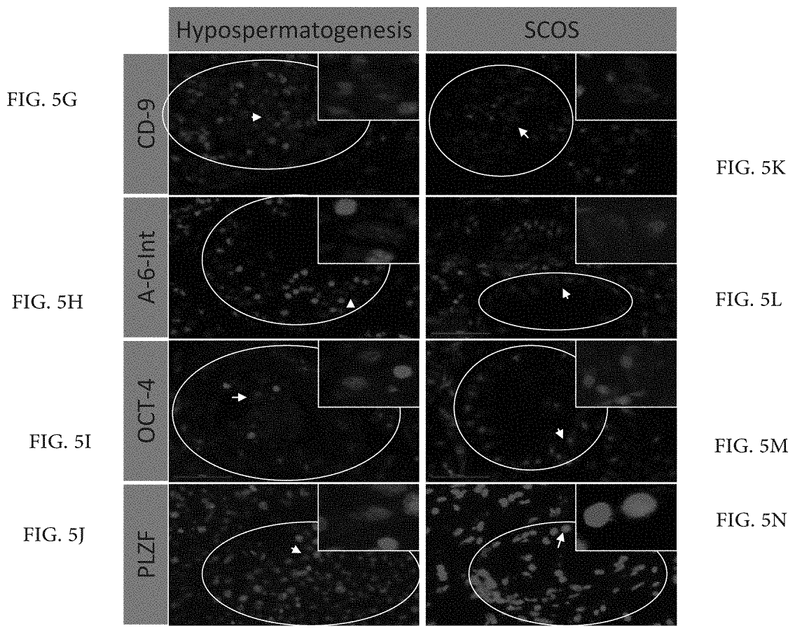

FIGS. 7A-E depict the morphology and summary of developed cell/colonies from isolated testicular cells in vitro. Germ cells from testicular biopsies of different azoospermic patients could form single cells and/or small, medium and large colonies in MCS culture. FIG. 7A—single cells;

FIG. 7B—pair cells; FIG. 7C—small colony (contained more than 10 cells and less than 50 cells); FIG. 7D—medium colony (contains between 50 and 150 cells); FIG. 7E—large colony (contains more than 150 cells).

FIGS. 8A-B are images depicting development of colonies from spermatogonial cells in MCS in a control medium alone (FIG. 8A; “CT”) or with the addition of TNFα (FIG. 8B; “TNF”).

FIGS. 9A-D depict the effect of hormones [testosterone (T) and FSH] and retinoic acid (RA) on the development of mouse spermatogenesis in vitro in methylcellulose culture system. Cells were enzymatically isolated from seminiferous tubules of ICR immature (7-day-old) mice. These cells were cultured in vitro (in methylcellulose culture system; 2×10⁵ cells/well/0.5 ml) in the presence of StemPro, KSR and growth factors (GDNF, LIF, EGF, bFGF) (CT—control medium). After two weeks of culture, various concentrations of testosterone (T; 10⁻⁶-10⁻⁸ M) (FIG. 9A) or retinoic acid (RA; 10⁻⁶-10⁻⁸ M) (FIG. 9C) were added to the cultures. After additional two weeks of culture, cells were collected and examined for VASA, CD9, CREM, BOULE and ACROSIN by qPCR analysis. FIGS. 9B and 9D—FSH was added to the culture from normal mice (FIG. 9B) or from busulfan-treated mice (FIG. 9D) from the beginning of the culture. The spermatogenic markers were examined after 4 weeks of the culture.

### DESCRIPTION OF SPECIFIC EMBODIMENTS OF THE INVENTION

The present invention, in some embodiments thereof, relates to methods of in vitro maturation of human spermatogonium.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Aggressive chemotherapy in childhood often results in testicular damage and consequently jeopardizes future fertility. The presence of spermatogonial cells (SPGCs) in the testes of prepubertal cancer patient boys (PCPBs) can be used to develop future strategies for male fertility preservation.

Spermatogenesis is a complicated process, which is composed of pre-meiotic, meiotic and post-meiotic stages. This process is developed in vivo in the seminiferous tubules of adult males. It is regulated by endocrine system and by testicular autocrine and paracrine factors that compose specific microenvironments for each stage of spermatogonial cell (SPGC) development to generate sperm. Recently, fertile sperm were generated from immature mouse using testicular organ culture system. Using three-dimension (3D) in vitro culture system, the development of sperm-like cells from SPGCs of immature mice, and the development of round spermatid from SPGCs of prepubertal monkeys were demonstrated. Induction of the proliferation of SPGCs in vitro from prepubertal cancer patients was demonstrated (Sadri-Ardekani H, Akhondi M A, van der Veen F, Repping S, van Pelt A M M. In vitro propagation of human prepubertal spermatogonial stem cells. J Am Med Asso 2011; 305: 2416-2418; Sadri-Ardekani H, Mizrak S C, van Daalen S K M, et al. Propagation of human spermatogonial stem cells in vitro. J Am Med Asso 2009; 19:2127-2134). However, the conditions to induce meiosis and post-meiotic stages of SPGCs from prepubertal cancer patient boys have not yet been described.

In the present study, the present inventor has examined the presence of SPGCs in testes of chemotherapy-treated PCPBs and their ability to develop spermatogenesis in vitro using a three-dimensional (3D) culture system. Seven testicular biopsies were obtained from chemotherapy-treated PCPBs and one from a patient with β-thalassemia major. Isolated testicular cells were cultured in a methylcellulose culture system (MCS)-containing STEMPRO® enriched with growth factors for 5-15 weeks. The presence of premeiotic, meiotic and postmeiotic cells was examined by immunofluorescence staining (IF) and/or RT-PCR analysis. The present inventor observed SPGCs in the examined testicular biopsies. Isolated testicular cells cultured in MCS developed into colonies and contained premeiotic, meiotic and postmeiotic cells. Furthermore, the present inventor identified sperm-like cells that had developed from testicular cells of a PCPB. These results demonstrate for the first time the presence of biologically active SPGCs in testicular biopsies of chemotherapy-treated PCPBs, and their capacity to develop in vitro to different stages of spermatogenesis including the generation of sperm-like cells. This study may open the way for new therapeutic strategies for fertility preservation of PCPBs and for azoospermic patients. The present inventor shows that testicular tissue from chemotherapy-treated cancer patient boys contain spermatogonial cells that could be induced to proliferate and to differentiate into meiotic and postmeiotic stages including the generation of sperm-like cells in 3D in vitro culture of MCS.

Thus, the present inventor has uncovered methods of inducing testicular germ cells from prepubertal cancer patient boys in vitro to meiotic and post-meiotic stages.

This is the first study that shows the expression of different pre-meiotic markers in testicular biopsies from prepubertal cancer patients after chemotherapy. In addition, this study shows for the first time a proof of concept for an in vitro culture system that induces human spermatogonial cells to meiotic and post-meiotic stages including the generation of sperm-like cells.

Thus, it was uncovered by the present inventor that testicular biopsies from chemotherapy-treated prepubertal cancer patient males contain biologically active spermatogonial cells. As shown herein, these cells could be induced in vitro using a three-dimensional culture system to complete spermatogenesis.

It should be noted that the methods of maturating human spermatogonium in vitro as described herein can be used along with the advanced intracytoplasmic sperm injection (ICSI) and in-vitro fertilization techniques to facilitate fertilization, thus can enable future reproduction of prepubertal boys that undergo aggressive chemo/radio therapy from their own germ line.

According to an aspect of some embodiments of the invention there is provided a method of in vitro maturation of human spermatogonium, comprising culturing the spermatogonium in a three-dimensional methylcellulose culture system (MCS) under conditions capable of differentiating the human spermatogonium into an elongated spermatid, thereby in vitro maturing the human spermatogonium.

As used herein the term “spermatogonium” refers to an undifferentiated male germ cell with a self-renewing capacity representing the first stage of spermatogenesis.

Spermatogonia undergo spermatogenesis to form mature spermatozoa in the seminiferous tubules of the testis. There are three major subtypes of spermatogonia in humans:

(i) Type A (dark) cells, with dark nuclei. These cells are reserve spermatogonial stem cells which do not usually undergo active mitosis;
(ii) Type A (pale) cells, with pale nuclei. These are the spermatogonial stem cells that undergo active mitosis. These cells divide to produce Type B cell;
(iii) Type B cells, which divide to give rise to primary spermatocytes.

As used herein the term “maturation” refers to the differentiation of a pre-meiotic spermatogonium into at least the meiotic, the post meiotic stage, and/or the mature elongated sperm.

It should be noted that each of the stages in spermatogonium maturation is characterized by typical morphological and/or molecular markers, such as cell surface expression markers.

The premeiotic spermatogonium stage includes several cells, such as type A cell which appears as a single cell (termed “As”), a type A cell that appears as a pair of two identical type A cells (termed “Apr”), a type A cell that appears as aligned cells (termed “Aal”), type A cells that are more differentiated (termed “A1-4”), intermediate stage cells (termed “In”), and a type B cell. Markers characteristics of As, Apr, Aal cells include, but are not limited to PLZF (promyelocytic leukaemia zinc finger), GFR-alpha1 (GDNF family receptor alpha-1), SALL4 (spalt like transcription factor 4), OCT4 (octamer-binding transcription factor 4), CD9 (CD9 molecule), alpha-6 integrin, and VASA [also known as “DEAD-box helicase 4 (DDX4)” or “MVH”). The expression of the marker C-kit (proto-oncogene receptor tyrosine kinase) is also characteristics of the most differentiating premeiotic cells. Markers characteristics of A 1-4, In and B cells include but are not limited to CD9, alpha-6 integrin, VASA, and C-kit.

The meiotic spermatogonium includes several typical cells, such as type spermatocyte (termed “SPC”) and round spermatid (termed “RS”). Markers characteristics of SPC include alpha-6 integrin, VASA, c-kit, LDH (Lactate Dehydrogenase), BOULE (boule homolog, RNA binding protein), CREM (cAMP responsive element modulator), and ACR (acrosin). Markers characteristics of RS include, but are not limited to VASA, c-KIT, LDH, BOUL, CREM, and ACR.

The post-meiotic spermatogonium includes the RS and elongated sperm (termed “ES”) cells. Markers characteristics of the RS cells include, but are not limited to VASA, LDH, BOUL, CREM, PROT (protamine), and ACR. Markers characteristics of the ES cells include, but are not limited to, the PROT and ACR markers.

A mature elongated sperm can be characterized by expression of at least one marker of the following cell surface markers: acrosin, and protamine. Morphological features of a meiotic spermatogonium include, for example, changes in the shape of the nucleus, and size of the cells (small).

According to some embodiments of the invention, the in vitro method of some embodiments of the invention results in maturation of the spermatogonium into a cell which expresses CREM.

According to some embodiments of the invention, the cell resulting from the method of maturation of some embodiments of the invention is characterized by the expression of CREM.

It should be noted that “CREM” (Gene ID: 1390), a cAMP responsive element modulator, is a bZIP transcription factor that binds to the cAMP responsive element found in many viral and cellular promoters. It is an important component of cAMP-mediated signal transduction during the spermatogenetic cycle, as well as other complex processes. Up to date, there is no evidence that human spermatogonia can be differentiated in vitro up to the stage of expressing the CREM-1 differentiation marker, using any known culturing system (e.g., either agar-based culturing system or methylcellulose based culturing system).

According to some embodiments of the invention, the human spermatogonium is an isolated human spermatogonium.

The term “isolated” refers to at least partially separated from the natural environment e.g., from a subject (e.g., human).

The human spermatogonium can be isolated from at least part of a testis tissue of a subject.

According to some embodiments of the invention, the human spermatogonium is comprised in a testicular biopsy of the subject.

According to some embodiments of the invention, the testicular biopsy is obtained from a prepubertal male subject.

According to some embodiments of the invention, the testicular biopsy is obtained from a non-obstructive azoospermic patient.

According to some embodiments of the invention, the testicular biopsy is a fresh tissue biopsy (removed from the testis of the subject).

According to some embodiments of the invention, the testicular biopsy is a frozen tissue biopsy, e.g., obtained by cryopreservation, e.g., as is further described hereinunder.

According to some embodiments of the invention, when using a frozen testicular biopsy, the testicular tissue biopsy is thawed to room temperature prior to culturing in the methylcellulose culture system of some embodiments of the invention.

Cryopreservation of testicular cells can be done by contacting a testicular tissue biopsy with a cryoprotectant (e.g., for 10 minutes in room temperature) and thereafter storing the tissue biopsy in liquid nitrogen for several months or years.

Briefly, for cryopreservation, after washing with a buffer such as PBS to remove residual blood, the biopsy is divided into small pieces, e.g., of about 3 mm³ each, and cryopreserved in 1.8 cryovials that contain 1.5 ml cryoprotectant media which can contain Dimethyl sulfoxide (DMSO) (e.g., about 5%), albumin (e.g., 10% human serum albumin) and sucrose (e.g., about 3.5% diluted in Hanks' Balanced Salt solution (HBSS)). The cooling rate of the biopsy can be gradual from about 37° C., through about 0° C. to about −80° C. For example, the cooling rate can be 0.5° C./minute, with holding at 0° C. for 9 minutes, followed by a cooling rate of 0.5° C./minute, until −8° C. with a holding of 5 minutes at this temperature. After 15 minutes holding at −8° C., the vials can be frozen to −40° C. at a rate of 0.5° C./min. The vials can then be frozen to −80° C. at a rate of 0.7° C./min and then transferred to liquid nitrogen. The cryopreserved biopsy can be thawed in room temperature (RT) and centrifuged (for washing) in the presence of any suitable culture medium such as Minimum Essential Media (MEM) (Biological Industries).

According to the method of some embodiments of the invention, culturing the spermatogonium is performed in a three-dimensional methylcellulose culture system (MCS).

The methylcellulose is used as a three-dimensional matrix or scaffold, to support the growth and/or differentiation (or maturation) of the spermatogonium.

As used herein the term “scaffold” or “matrix”, which are interchangeably used herein, refers to a two-dimensional or a three-dimensional supporting framework.

According to some embodiments of the invention, the scaffold is a three-dimensional scaffold.

According to some embodiments of the invention, the scaffold enables the proliferation and/or differentiation of the spermatogonium into at least the meiotic and/or post-meiotic stage and/or a mature sperm.

According to some embodiments of the invention, the scaffold is a methylcellulose scaffold.

Methylcellulose is a synthetic (non-natural) chemical compound derived from cellulose. It is available as a hydrophilic white powder, preferably in a pure form which can be dissolved in cold water, forming a clear viscous solution or gel, or in an already dissolved ready to use viscous solution. Methylcellulose is synthetically produced by heating cellulose with caustic solution (e.g. a solution of sodium hydroxide) and treating it with methyl chloride. In the substitution reaction that follows, the hydroxyl residues (—OH functional groups) are replaced by methoxide (—OCH3 groups).

Different kinds of methylcellulose can be prepared depending on the number of hydroxyl groups substituted. Cellulose is a polymer consisting of numerous linked glucose molecules, each of which exposes three hydroxyl groups. The Degree of Substitution (DS) of a given form of methylcellulose is defined as the average number of substituted hydroxyl groups per glucose. The theoretical maximum is thus a DS of 3.0, however more typical values are 1.3-2.6. Different methylcellulose preparations can also differ in the average length of their polymer backbones. The methylcellulose can be obtained from various suppliers such as R&D, Minneapolis, USA, and Sigma-Aldrich.

Suitable concentrations of methylcellulose matrixes include from about 35% to about 50% (volume/volume), e.g., between 40-50% (v/v), e.g., between 40-45% (v/v), e.g., at a concentration of about 42% (v/v).

According to some embodiments of the invention, the cells are directly added into a culture system which includes a suitable culture medium and a methylcellulose matrix.

Additionally or alternatively, the cells which are seeded onto the methylcellulose culture system (the MC matrix and culture medium) are mainly non-adherent cells. It should be noted that the present inventor has uncovered that seeding of adherent cells in the MCS is less efficient than seeding of isolated cells which are mainly (e.g., more than 50%, more than 60%, more than 70%, more than 80%, more than 90%) non-adherent cells.

According to some embodiments of the invention, prior to culturing in the methylcellulose culture systems (MCS) the cells are cultured in a culture medium under conditions which enable removal of adherent cells, while isolating at least 50%, at least 60%, at least 70%, at least 80%, at least 90%, at least 95% or more of the non-adherent cells. For example, the cells of the testis tissue biopsy are cultured in a tissue culture plate, such as a multi-well plate, e.g., a 24-well plate, preferably in uncoated wells of a tissue-culture grade flask. The cells are seeded at a concentration of about 4-5×10⁴ cells/well/500 μl, e.g., at about 2×10⁴ cells/well/500 μl in a culture medium (base medium) such as MEM medium which improves adherence of adherent cells to the plastic. A suitable basic culture medium may comprise sodium bicarbonate (e.g., at a concentration of 7.5%), L-glutamine (e.g., at a concentration of 200 mM), non-essential amino acids (e.g., at a concentration of 1%), penicillin/streptomycin and gentamicin (e.g., at a concentration of 10 mg/ml), and incubated for about 2 nights at 37° C., 5% CO2.

The nonadherent cells are then collected and cultured (at a concentration of about 2-5×10⁴ cells/well/500 μl; or at a concentration of 4-5×10⁴ cells/well/500 μl) in methylcellulose as a three-dimensional (3D) culture system.

The cells are usually diluted in a culture medium prior to the addition of the culture medium onto the methylcellulose scaffold. For example, if 42% of methylcellulose is used, then the cells are diluted in the remaining 58% of culture medium. However, it is appreciated that the cells can be also added to an already mixed medium and methylcellulose culture system.

The culture medium used in the MC culture system can include a base medium supplemented with serum and/or serum replacement, as described hereinunder. For example, in the case of using 42% methylcellulose matrix, the cells can be diluted in the remaining 58% of culture medium which can comprise 33% StemPro-34 medium and 25% KSR (knock-out serum replacement) (Gibco, USA) enriched with different factors and reagents described herein below.

Alternatively or additionally, the cells can be diluted in a medium which contains 33% StemPro-34 medium (Gibco, e.g., from USA) and the StemPro supplement (e.g., at a concentration of 2.6%; e.g., from Gibco), and optionally with the addition of insulin (e.g., at a concentration of 25 μg/ml; e.g., from Gibco), transferrin (e.g., at a concentration of 100 μg/ml; e.g., from Gibco), putrescin (e.g., at a concentration of 60 μg/ml; Gibco), sodium selenite (e.g., at a concentration of 30 nM; e.g., from Gibco), D-glucose (e.g., at a concentration of 6 mg/ml; e.g., from Sigma), pyruvic acid (e.g., at a concentration of 30□ μg/ml; e.g., from Sigma), bovine serum albumin (BSA) (e.g., at a concentration of 5 mg/ml; Millpore, Illkirch, France), L-glutamine (e.g., at a concentration of 2 mM; e.g., from Biological Industries), 2-mercaptoethanol (e.g., at a concentration of 0.5 μM; e.g., from Gibco), MEM vitamin solution (e.g., at a concentration of 10 μl/ml; e.g., from Gibco, UK), MEM non-essential amino acid solution (e.g., at a concentration of 10 μl/ml; e.g., from Gibco, UK), ascorbic acid (e.g., at a concentration of 100 μM; e.g., from Sigma, China), d-biotin (e.g., at a concentration of 10 μg/ml; e.g., from Sigma), KSR (e.g., at a concentration of 1%, e.g., from Gibco, UK), Pen/Strep (e.g., from Biological Industries), enriched with different factors as described hereinunder.

For establishment of the methylcellulose (MC) culture system, a culture medium containing the isolated cells from the testicular biopsy (e.g., 58% final dilution in the well) are mixed with MC (e.g., 42% final dilution in the well) and are cultured in the wells. Cells were cultured for 1-16 weeks, e.g., for about 5-15 weeks in CO2 incubator at 37° C.

Every 7-14 days a fresh concentrated medium (X10) can be added, e.g., 50 μl/well of fresh concentrated (×10) enriched StemPro-34 medium (containing all the growth factors used in the primary culture) to the cell cultures to be followed up after additional 1-2 weeks.

As described, the culture medium used for culturing the spermatogonium comprises various agents, growth factors and/or hormones.

According to some embodiments of the invention, the conditions comprise culturing the human spermatogonium in a culture medium which comprises an effective concentration of at least one growth factor selected from the group consisting of Glial cell line-derived neurotrophic factor (GDNF), leukemia inhibitory factor (LIF), basic fibroblast growth factor (bFGF), and epidermal growth factor (EGF).

According to some embodiments of the invention, the culture medium further comprises an effective concentration of TNFalpha (TNFα).

According to some embodiments of the invention, the culture medium further comprises an effective concentration of at least one agent selected from the group consisting of: testosterone, follicle stimulating hormone (FSH) and retinoic acid.

According to some embodiments of the invention, the method further comprising culturing the human spermatogonium in the presence of at least one hormone selected from the group consisting of: Follicle-Stimulating Hormone (FSH) and testosterone.

According to some embodiments of the invention, the conditions comprise culturing said human spermatogonium in a culture medium which comprises an effective concentration of at least one growth factor selected from the group consisting of TNFalpha (TNFα), Glial cell line-derived neurotrophic factor (GDNF), leukemia inhibitory factor (LIF) and basic fibroblast growth factor (bFGF).

According to some embodiments of the invention, the conditions comprise culturing the human spermatogonium in a culture medium which comprises an effective concentration of at least one growth factor selected from the group consisting of GDNF, LIF, and bFGF.

According to some embodiments of the invention, the conditions comprise culturing the human spermatogonium in a culture medium which comprises an effective concentration of at least one growth factor selected from the group consisting of GDNF, LIF, and EGF.

According to some embodiments of the invention, the conditions comprise culturing the human spermatogonium in a culture medium which comprises an effective concentration of at least one growth factor selected from the group consisting of GDNF, bFGF, and EGF.

According to some embodiments of the invention, the conditions comprise culturing the human spermatogonium in a culture medium which comprises an effective concentration of at least one growth factor selected from the group consisting of LIF, bFGF, and EGF.

According to some embodiments of the invention, the conditions comprise culturing said human spermatogonium in a culture medium which comprises an effective concentration of Glial cell line-derived neurotrophic factor (GDNF).

Glial cell line-derived neurotrophic factor (GDNF) is a secreted ligand of the TGF-beta (transforming growth factor-beta) superfamily of proteins. Ligands of this family bind various TGF-beta receptors leading to recruitment and activation of SMAD family transcription factors that regulate gene expression. GDNF can be provided from various suppliers and makers, such as from Biolegend (CA, USA), PROSPEC Protein Specialists (Rehovot, Ill.), and ThermoFisher Scientific.

According to some embodiments of the invention, the effective concentration of GDNF is in the range of 1-50 ng/ml, e.g., 1-40 ng/ml, e.g., 1-30 ng/ml, e.g., 1-25 ng/ml, e.g., 1-20 ng/ml, e.g., about 5 ng/ml, e.g., about 10 ng/ml, e.g., about 15 ng/ml, e.g., about 20 ng/ml.

According to some embodiments of the invention, the conditions comprise culturing said human spermatogonium in a culture medium which comprises an effective concentration of leukemia inhibitory factor (LIF).

Leukemia inhibitory factor (LIF) is a pleiotropic cytokine with roles in several different systems. It is involved in the induction of hematopoietic differentiation in normal and myeloid leukemia cells, induction of neuronal cell differentiation, regulator of mesenchymal to epithelial conversion during kidney development, and may also have a role in immune tolerance at the maternal-fetal interface. LIF can be provided from various suppliers and makers, such as from Biolegend (CA, USA), PEPROTECH® (Rehovot, Ill.), and Sigma-Aldrich® (MERCK).

According to some embodiments of the invention, the effective concentration of LIF is in the range of 1-50 ng/ml, e.g., 1-40 ng/ml, e.g., 1-30 ng/ml, e.g., 1-25 ng/ml, e.g., 1-20 ng/ml, e.g., about 5 ng/ml, e.g., about 10 ng/ml, e.g., about 15 ng/ml, e.g., about 20 ng/ml.

According to some embodiments of the invention, the conditions comprise culturing said human spermatogonium in a culture medium which comprises an effective concentration of basic fibroblast growth factor (bFGF).

Basic fibroblast growth factor (bFGF) is a member of the fibroblast growth factor (FGF) family. FGF family members bind heparin and possess broad mitogenic and angiogenic activities. This protein has been implicated in diverse biological processes, such as limb and nervous system development, wound healing, and tumor growth, bFGF can be provided from various suppliers and makers, such as from Biolegend (CA, USA), and Invitrogen Corporation products (Grand Island N.Y., USA).

According to some embodiments of the invention, the effective concentration of bFGF is in the range of 1-50 ng/ml, e.g., 1-40 ng/ml, e.g., 1-30 ng/ml, e.g., 1-25 ng/ml, e.g., 1-20 ng/ml, e.g., about 5 ng/ml, e.g., about 10 ng/ml, e.g., about 15 ng/ml, e.g., about 20 ng/ml.

According to some embodiments of the invention, the conditions comprise culturing said human spermatogonium in a culture medium which comprises an effective concentration of epidermal growth factor (EGF).

Epidermal growth factor (EGF) is a member of the epidermal growth factor superfamily. EGF acts a potent mitogenic factor that plays an important role in the growth, proliferation and differentiation of numerous cell types. This protein acts by binding with high affinity to the cell surface receptor, epidermal growth factor receptor. Defects in the EGF gene are the cause of hypomagnesemia type 4, and dysregulation of the EGF gene has been associated with the growth and progression of certain cancers. Alternative splicing results in multiple transcript variants, at least one of which encodes a preproprotein that is proteolytically processed.

EGF can be provided from various suppliers and makers, such as from Biolegend (CA, USA), ALMONE LABS (Hadassah Ein Kerem, Ill.), PROSPEC Protein Specialists (Rehovot, Ill.), and ACRO BIOSYSTEMS (St. Louis, Mo., USA). According to some embodiments of the invention, the effective concentration of EGF is in the range of 0.1-200 ng/ml, e.g., 0.5-100 ng/ml, e.g., 1-80 ng/ml, e.g., 1-50 ng/ml, e.g., 5-50 ng/ml, e.g., about 5-25 ng/ml, e.g., between 18-25 ng/ml, e.g., between 19-21 ng/ml, e.g., about 5 ng/ml, 10 ng/ml, e.g., about 15 ng/ml, e.g., about 20 ng/ml, e.g., about 20 ng/ml (e.g., 20 ng/ml).

According to some embodiments of the invention, the conditions comprise culturing said human spermatogonium in a culture medium which comprises an effective concentration of TNFalpha (TNFα).

Tumor necrosis factor (TNF) alpha (TNFα) is a multifunctional proinflammatory cytokine that belongs to the tumor necrosis factor (TNF) superfamily. This cytokine is mainly secreted by macrophages and can bind to the TNFRSF1A/TNFR1 and TNFRSF1B/TNFBR receptors. TNFα can be provided from various suppliers and makers, such as from ALMONE LABS (Hadassah Ein Kerem, Ill.), PROSPEC Protein Specialists (Rehovot, Ill.), and Biolegend, CA, USA.

According to some embodiments of the invention, the effective concentration of TNFalpha (TNFα) is in the range of 1-200 pg/ml, e.g., 1-100 pg/ml, e.g., 1-50 pg/ml, e.g., about 5 pg/ml, e.g., about 10 pg/ml, e.g., about 15 pg/ml, e.g., about 20 pg/ml, e.g., about 25 pg/ml, e.g., about 30 μg/ml, e.g., about 35 pg/ml, e.g., about 40 pg/ml, e.g., about 50 pg/ml.

Testosterone is the primary male sex hormone and an anabolic steroid from the androstane class containing a keto and hydroxyl groups at the three and seventeen positions respectively. It is biosynthesized in several steps from cholesterol and is converted in the liver to inactive metabolites. It exerts its action through binding to and activation of the androgen receptor. In male humans, testosterone plays a key role in the development of male reproductive tissues such as testes and prostate, as well as promoting secondary sexual characteristics. Testosterone can be obtained from various sources and suppliers such as Sigma-Aldrich® (MERCK).

According to some embodiments of the invention, the effective concentration of testosterone is in the range of 1×10⁻⁸ M (molar) through 1×10⁻⁶ M, e.g., 1×10⁻⁸ M, e.g., 1×10⁻⁷ M, e.g., 1×10⁻⁶ M.

Follicle stimulating hormone (FSH) is a gonadotropin, a glycoprotein polypeptide hormone, which is synthesized and secreted by the gonadotropic cells of the anterior pituitary gland, and regulates the development, growth, pubertal maturation, and reproductive processes of the body.

FSH can be obtained from various sources and suppliers such as Sigma-Aldrich® (MERCK).

According to some embodiments of the invention, the effective concentration of FSH is in the range of 1 U/ml (unit per milliliter) to 100 U/ml, e.g., between 5-50 U/ml, e.g., between 10-50 U/ml, e.g., between 20-40 U/ml, e.g., between 20-30 U/ml, e.g., 25 U/ml.

Retinoic acid is a metabolite of vitamin A (retinol) that mediates the functions of vitamin A required for growth and development. During early embryonic development, retinoic acid generated in a specific region of the embryo helps determine position along the embryonic anterior/posterior axis by serving as an intercellular signaling molecule that guides development of the posterior portion of the embryo.

Retinoic acid can be obtained from various sources and suppliers such as Sigma-Aldrich® (MERCK).

According to some embodiments of the invention, the effective concentration of retinoic acid is in the range of 1×10⁻⁸ M (molar) through 1×10⁻⁶ M, e.g., 1×10⁻⁸ M, e.g., 1×10⁻⁷ M, e.g., 1×10⁻⁶M.

According to some embodiments of the invention, the at least one hormone is added in the beginning of the culturing process, along with the at least one growth factor of the growth factors described hereinabove.

According to some embodiments of the invention, the culturing in the presence of the at least one hormone is performed following about one month of culturing in the presence of the at least one growth factor, e.g., following about two months of culturing in the presence of said at least one growth factor.

According to some embodiments of the invention, the at least one hormone is added to the culture medium which comprises the at least one growth factor.

According to some embodiments of the invention, the at least one hormone is added to a culture medium which comprises at least one growth factor from the growth factors selected from the group consisting of: GDNF, LIF, bFGF, and EGF.

According to some embodiments of the invention, the at least one hormone is added to a culture medium which comprises at least 2 growth factors (e.g., at least 2 factors from the growth factors selected from the group consisting of: GDNF, LIF, bFGF, and EGF).

According to some embodiments of the invention, the at least one hormone is added to a culture medium which comprises at least 3 growth factors (e.g., at least 3 growth factors from the growth factors selected from the group consisting of: GDNF, LIF, bFGF, and EGF).

According to some embodiments of the invention, the at least one hormone is added to a culture medium which comprises at least 4 growth factors (e.g., at least GDNF, LIF, bFGF, and EGF).

As used herein the phrase “culture medium” refers to a liquid substance used to support the growth of spermatogonial cells and optionally induce their proliferation and/or differentiation to meiotic and postmeiotic stages including the generation of sperm-like cells in 3D in vitro culture of MCS. The culture medium used according to some embodiments of the invention can be a water-based medium which includes a combination of substances such as salts, nutrients, minerals, vitamins, amino acids, nucleic acids, and/or proteins such as cytokines, growth factors and hormones, all of which are needed for cell proliferation and/or differentiation of spermatogonial cells into meiotic and postmeiotic stages including the generation of sperm-like cells in 3D in vitro culture of MCS.

For example, a culture medium according to an aspect of some embodiments of the invention can be a synthetic tissue culture medium such as the StemPro® (Thermo Fisher Scientific), Ko-DMEM (Gibco-Invitrogen Corporation products, Grand Island, N.Y., USA), DMEM/F12 (Biological Industries, Biet HaEmek, Israel), RPMI (Biological Industries, Biet HaEmek, Israel), supplemented with the necessary additives as is further described herein.

According to some embodiments of the invention, the culture medium comprises serum replacement.

Serum replacement is usually added to most culture media which are designed for culturing stem cells or progenitor cells, in order to provide the cells with the optimal environment, similar to that present in vivo (i.e., within the organism from which the cells are derived, e.g., for a developing spermatogonium in the testis).

Serum replacement is used in culture media to replace the need of a serum. While the use of serum which is derived from either an animal source (e.g., bovine serum) or a human source (human serum) is limited by the significant variations in serum components between individuals and the risk of having xeno contaminants (in case of an animal serum is used), the use of the more defined composition such as the currently available serum Replacement™ (Gibco-Invitrogen Corporation, Grand Island, N.Y. USA) may be limited by the presence of Albumax (Bovine serum albumin enriched with lipids) which is from an animal source within the composition (International Patent Publication No. WO 98/30679 to Price, P J. et al).

Various animal-free formulations of serum replacement are available for in vitro culturing.

According to some embodiments of the invention, the culture medium comprises STEM PRO® (Thermo Fisher Scientific) supplement.

The StemPro® (Thermo Fisher Scientific) hESC SFM (serum free medium) is a fully-defined serum- and feeder-free medium specifically formulated for the growth and expansion of human embryonic stem cells (hESCs). The StemPro® (Thermo Fisher Scientific) hESC SFM includes DMEM/F-12 with GlutaMAX™ (Thermo Fisher Scientific) medium, StemPro® hESC Supplement and Bovine serum albumin 25% (BSA). The Gibco™ GlutaMAX™ media contains a stabilized form of L-glutamine, L-alanyl-L-glutamine, preventing degradation and ammonia build-up even during long-term cultures.

It should be noted that when an animal-contaminant-free serum replacement is used to culture human cells, then the serum replacement is referred to as being “xeno-free”.

The term “xeno” is a prefix based on the Greek word “Xenos”, i.e., a stranger. As used herein the phrase “xeno-free” refers to being devoid of any components/contaminants which are derived from a xenos (i.e., not the same, a foreigner) species.

For example, a xeno-free serum replacement for use with human cells (i.e., an animal contaminant-free serum replacement) can include a combination of insulin, transferrin and selenium. Additionally or alternatively, a xeno-free serum replacement can include human or recombinantly produced albumin, transferrin and insulin.

Non-limiting examples of commercially available xeno-free serum replacement compositions include the premix of ITS (Insulin, Transferrin and Selenium) available from Invitrogen corporation (ITS, Invitrogen, Catalogue No. 51500-056); Serum replacement 3 (SR3; Sigma, Catalogue No. S2640) which includes human serum albumin, human transferring and human recombinant insulin and does not contain growth factors, steroid hormones, glucocorticoids, cell adhesion factors, detectable Ig and mitogens; KnockOut™ SR XenoFree [Catalogue numbers A10992-01, A10992-02, part Nos. 12618-012 or 12618-013, Invitrogen GIBCO] which contains only human-derived or human recombinant proteins.

According to some embodiments of the invention, the ITS (Invitrogen corporation) or SR3 (Sigma) xeno-free serum replacement formulations are diluted in a 1 to 100 ratio in order to reach a ×1 working concentration.

According to some embodiments of the invention, the concentration of the serum replacement [e.g., KnockOut™ SR XenoFree (Invitrogen)] in the culture medium is in the range of from about 1% [volume/volume (v/v)] to about 50% (v/v), e.g., from about 5% (v/v) to about 40% (v/v), e.g., from about 5% (v/v) to about 30% (v/v), e.g., from about 10% (v/v) to about 30% (v/v), e.g., from about 10% (v/v) to about 25% (v/v), e.g., from about 10% (v/v) to about 20% (v/v), e.g., about 10% (v/v), e.g., about 15% (v/v), e.g., about 20% (v/v), e.g., about 30% (v/v), e.g., about 25% (v/v).

Once in the MCS, the cells can be evaluated for their differentiation state. According to some embodiments of the invention, the cells in the MCS are evaluated every 10 days to 2 weeks under the microscope for the growth quality/viability and the morphology of the developed colonies.

It should be noted that about 10% of the cells may present apoptotic vacuoles in their cytoplasm.

According to some embodiments of the invention, the cells are cultured in the MC systems for about 3 months, in order to mimic the physiological timing of development of human spermatogenesis (around 3 months).

At the end of the incubation period in MCS, the cells can be collected by adding a buffer (e.g., PBS, e.g., an amount of 0.5 ml PBS to each culture well that contained 0.5 ml MC mix), further pipetting the buffer and collecting the suspension to a new tube (e.g., a 15 ml tube). The tubes are centrifuged (e.g., in 1600 RPM for 10 minutes) to remove the excess of culture medium and buffer. Most of the volume is removed and the remainder liquid (around 100 μl from the bottom of the tube) is collected. This volume which contains the differentiated cells, can be isolated and be further used for clinical purposes.

Additionally or alternatively, the differentiated cells can be smeared on a slide for further evaluations (e.g., histological evaluations), and/or can be collected and kept at −70° C. (to be used for RNA analyses). In case there are more than 10-15 small colonies, or 5 medium or large colonies in the well, the cells can be utilized for both IF and RNA analyses.

The fertilization capacity and epigenetics of the generated post-meiotic and sperm-like cells can be evaluated. Methods of qualifying the degree of meiotic differentiating are known in the art and include, for example, monitoring the expression levels of various pre-meiotic (e.g., VASA, SALL4, OCT4, PLZF, CD9, A-6-INTEGRIN, GFR-A1, and C-KIT), meiotic (e.g., CREM-1, LDH, ACROSIN, and BOULE) and post-meiotic (e.g., ACROSIN, and PROTAMINE) markers. Such methods include RNA or protein detection methods which are well known in the art. Non-limiting examples of such methods and exemplary antibodies and/or primers for RNA analysis are described hereinunder.

According to some embodiments of the invention, the method further comprises identifying a meiotic cell, a post meiotic cell and/or a mature sperm cell resulting from the in vitro maturation.

According to some embodiments of the invention, identification of the meiotic cell, the post meiotic cell and/or the mature sperm cell is by a characteristic marker to the meiotic cell, the post meiotic cell and/or the mature sperm cell, respectively.

According to some embodiments of the invention, the method further comprises identifying a cell expressing CREM resulting from the in vitro maturation of the spermatogonium.

According to some embodiments of the invention, the method further comprises identifying a cell expressing acrosin resulting from the in vitro maturation of the spermatogonium.

According to some embodiments of the invention, the method further comprises identifying a sperm cell (elongated cell) resulting from the in vitro maturation of the spermatogonium using a mitochondrial staining (e.g., MitoTracker). The resulting sperm cell has a concentrated mitochondria in the neck which intensely stains in green.

The advantage of using MitoTracker is to identify a few sperm cells that could be used for in vitro fertilization since the MitoTracker stains live cells without fixation.

According to an aspect of some embodiments of the invention there is provided an in vitro matured sperm obtainable according to the method of some embodiments of the invention.

According to an aspect of some embodiments of the invention there is provided a method of treating a subject in need of mature sperm cells, comprising:

(a) obtaining a spermatogonium from the subject, and

(b) subjecting said spermatogonium to an in vitro maturation according to the method of some embodiments of the invention, thereby generating mature sperm cells of the subject, and treating the subject.

According to some embodiments of the invention, the subject is a prepubertal male subject.

According to some embodiments of the invention, the prepubertal male subject is in need of aggressive chemotherapy and/or aggressive radiotherapy.

According to some embodiments of the invention, the prepubertal male subject is diagnosed with cancer.

According to some embodiments of the invention the cancer can be a solid tumor or a non-solid cancer and/or cancer metastasis.

According to some embodiments of the invention, the cancer comprises a solid tumor.

According to some embodiments of the invention, the cancer comprises a hematological cancer.

Examples of cancer include, but are not limited to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, testicular germ cells tumor, sacrococcygeal tumor, choriocarcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

According to some embodiments of the invention, the prepubertal male subject is diagnosed with an autoimmune disease.

Autoimmune diseases include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel el al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March, 92 (1):77), spontaneous autoimmune thyroiditis (Braley-Mullen H. and Yu S, J Immunol 2000 Dec. 15; 165 (12):7262), Hashimoto's thyroiditis (Toyoda N. el al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 Febuary; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1): 16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. el al., Clin Immunol Immunopathol 1990 March, 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April, 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

According to some embodiments of the invention, the prepubertal male subject is diagnosed with thalassemia (β-thalassemia major).

According to some embodiments of the invention, the prepubertal male subject is in need of bone marrow transplantation.

According to some embodiments of the invention, the subject is a non-obstructive azoospermic patient.

According to some embodiments of the invention, the subject is in need of aggressive chemotherapy and/or aggressive radiotherapy.

It should be noted that the testis tissue biopsy can be obtained from the subject using known surgical methods before administering the suitable anti-cancer drug(s) or the radiation therapy to the subject. In this case, the tissue biopsy can be cryopreserved and the tissue can be kept frozen until future use. Then after, upon thawing the frozen biopsy, the isolated spermatogonia are subjected to the in vitro maturation method of some embodiments of the invention.

Additionally or alternatively, the testis tissue biopsy can be obtained from the subject during or after treating the subject with anti-cancer drug(s) (e.g., chemotherapy) or radiation. In this case, the tissue biopsy can be used as either a fresh biopsy for isolation of spermatogonia cells that are cultured in the methylcellulose culture system according to the method of some embodiments of the invention, or can be cryopreserved and kept frozen until future use. Then after, upon thawing the frozen biopsy, the isolated spermatogonia are subjected to the in vitro maturation method of some embodiments of the invention.

As mentioned, qualifying the maturation stage following the in vitro culturing of the spermatogonium according to the method of some embodiments of the invention, can be done by detecting the expression of the markers typical to each cell type of the spermatogenesis, e.g., meiotic, post meiotic and mature elongated sperm cell. Such markers are known in the art, and are also described herein above. Following is a non-limiting example of primary antibodies can be used to detect the spermatogenesis-specific markers:

(i) monoclonal mouse anti-human PLZF (promyelocytic leukemia zinc finger). Such antibodies are available for example from Santa Cruz;

(ii) Polyclonal goat anti-human GFR-α (GDNF family receptor alpha-1). Such antibodies are available for example from R&D, MN, USA;

(iii) Rabbit Polyclonal to Human SALL4 (spalt like transcription factor 4). Such antibodies are available for example from LSBio LifeSpan BioSciences, Inc. Seattle Wash., USA;

(iv) Polyclonal rabbit anti-human CD9 (CD9 molecules). Such antibodies are available for example from Abcam, Cambridge, UK;

(v) Polyclonal goat anti-human OCT4 (octamer-binding transcription factor 4). Such antibodies are available for example from Santa Cruz;

(vi) Polyclonal rabbit anti-human α-6-INTEGRIN. Such antibodies are available for example from Santa Cruz;

(vii) polyclonal rabbit anti-human VASA [also known as “DEAD-box helicase 4 (DDX4)” or “MVH”). Such antibodies are available for example from Santa Cruz, Calif., USA;

(viii) Polyclonal rabbit anti-human c-KIT (proto-oncogene receptor tyrosine kinase). Such antibodies are available for example from Dako, CA, USA;

Exemplary secondary antibodies which can be used include, but are not limited to, Donkey anti-rabbit IgG (Cy3), Donkey anti-goat IgG (Cy3), and Goat anti-mouse IgG (Rhodamine red) Jackson Immuno Research (USA).

Non-limiting examples of suitable RT-PCR primers, which can be sued for detection of the spermatogenesis specific markers are provided in SEQ ID NOs: 1 and 2 (for OCT4); SEQ ID NOs: 3 and 4 (for SALL4); SEQ ID NOs: 5 and 6 (for alpha 6 integrin); SEQ ID NOs: 7 and 8 (for CD9); SEQ ID NOs: 9 and 10 (for GFR alpha-1); SEQ ID NOs: 11 and 12 (for c-KIT); SEQ ID NOs: 13 and 14 (for CREM); SEQ ID NOs: 15 and 16 (for protamine);

Methods of Detecting the Expression Level of RNA

The expression level of the RNA in the cells of some embodiments of the invention can be determined using methods known in the arts.

Northern Blot Analysis:

This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR Analysis:

This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

RNA In Situ Hybridization Stain:

In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the bound probe is detected using known methods. For example, if a radio-labeled probe is used, then the slide is subjected to a photographic emulsion which reveals signals generated using radio-labeled probes; if the probe was labeled with an enzyme then the enzyme-specific substrate is added for the formation of a colorimetric reaction; if the probe is labeled using a fluorescent label, then the bound probe is revealed using a fluorescent microscope; if the probe is labeled using a tag (e.g., digoxigenin, biotin, and the like) then the bound probe can be detected following interaction with a tag-specific antibody which can be detected using known methods.

In Situ RT-PCR Stain:

This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, Calif.).

DNA Microarrays/DNA Chips:

The expression of thousands of genes may be analyzed simultaneously using DNA microarrays, allowing analysis of the complete transcriptional program of an organism during specific developmental processes or physiological responses. DNA microarrays consist of thousands of individual gene sequences attached to closely packed areas on the surface of a support such as a glass microscope slide. Various methods have been developed for preparing DNA microarrays. In one method, an approximately 1 kilobase segment of the coding region of each gene for analysis is individually PCR amplified. A robotic apparatus is employed to apply each amplified DNA sample to closely spaced zones on the surface of a glass microscope slide, which is subsequently processed by thermal and chemical treatment to bind the DNA sequences to the surface of the support and denature them. Typically, such arrays are about 2×2 cm and contain about individual nucleic acids 6000 spots. In a variant of the technique, multiple DNA oligonucleotides, usually 20 nucleotides in length, are synthesized from an initial nucleotide that is covalently bound to the surface of a support, such that tens of thousands of identical oligonucleotides are synthesized in a small square zone on the surface of the support. Multiple oligonucleotide sequences from a single gene are synthesized in neighboring regions of the slide for analysis of expression of that gene. Hence, thousands of genes can be represented on one glass slide. Such arrays of synthetic oligonucleotides may be referred to in the art as “DNA chips”, as opposed to “DNA microarrays”, as described above [Lodish et al. (eds.). Chapter 7.8: DNA Microarrays: Analyzing Genome-Wide Expression. In: Molecular Cell Biology, 4th ed., W. H. Freeman, New York. (2000)].

Oligonucleotide Microarray—

In this method oligonucleotide probes capable of specifically hybridizing with the polynucleotides of some embodiments of the invention are attached to a solid surface (e.g., a glass wafer). Each oligonucleotide probe is of approximately 20-25 nucleic acids in length. To detect the expression pattern of the polynucleotides of some embodiments of the invention in a specific cell sample (e.g., blood cells), RNA is extracted from the cell sample using methods known in the art (using e.g., a TRIZOL solution, Gibco BRL, USA). Hybridization can take place using either labeled oligonucleotide probes (e.g., 5′-biotinylated probes) or labeled fragments of complementary DNA (cDNA) or RNA (cRNA). Briefly, double stranded cDNA is prepared from the RNA using reverse transcriptase (RT) (e.g., Superscript II RT), DNA ligase and DNA polymerase I, all according to manufacturer's instructions (Invitrogen Life Technologies, Frederick, Md., USA). To prepare labeled cRNA, the double stranded cDNA is subjected to an in vitro transcription reaction in the presence of biotinylated nucleotides using e.g., the BioArray High Yield RNA Transcript Labeling Kit (Enzo, Diagnostics, Affymetix Santa Clara Calif.). For efficient hybridization the labeled cRNA can be fragmented by incubating the RNA in 40 mM Tris Acetate (pH 8.1), 100 mM potassium acetate and 30 mM magnesium acetate for 35 minutes at 94° C. Following hybridization, the microarray is washed and the hybridization signal is scanned using a confocal laser fluorescence scanner which measures fluorescence intensity emitted by the labeled cRNA bound to the probe arrays.

For example, in the Affymetrix microarray (Affymetrix®, Santa Clara, Calif.) each gene on the array is represented by a series of different oligonucleotide probes, of which, each probe pair consists of a perfect match oligonucleotide and a mismatch oligonucleotide. While the perfect match probe has a sequence exactly complimentary to the particular gene, thus enabling the measurement of the level of expression of the particular gene, the mismatch probe differs from the perfect match probe by a single base substitution at the center base position. The hybridization signal is scanned using the Agilent scanner, and the Microarray Suite software subtracts the non-specific signal resulting from the mismatch probe from the signal resulting from the perfect match probe.

Methods of Detecting Expression and/or Activity of Proteins

Expression and/or activity level of proteins expressed in the cells of some embodiments of the invention can be determined using methods known in the arts.

Enzyme Linked Immunosorbent Assay (ELISA):

This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western Blot:

This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-Immunoassay (RIA):

In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence Activated Cell Sorting (FACS):

This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical Analysis:

This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxylin or Giemsa stain.

In Situ Activity Assay:

According to this method, a chromogenic substrate is applied on the cells containing an active enzyme and the enzyme catalyzes a reaction in which the substrate is decomposed to produce a chromogenic product visible by a light or a fluorescent microscope.

In Vitro Activity Assays:

In these methods the activity of a particular enzyme is measured in a protein mixture extracted from the cells. The activity can be measured in a spectrophotometer well using colorimetric methods or can be measured in a non-denaturing acrylamide gel (i.e., activity gel). Following electrophoresis the gel is soaked in a solution containing a substrate and colorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy.

Analysis and Discussion

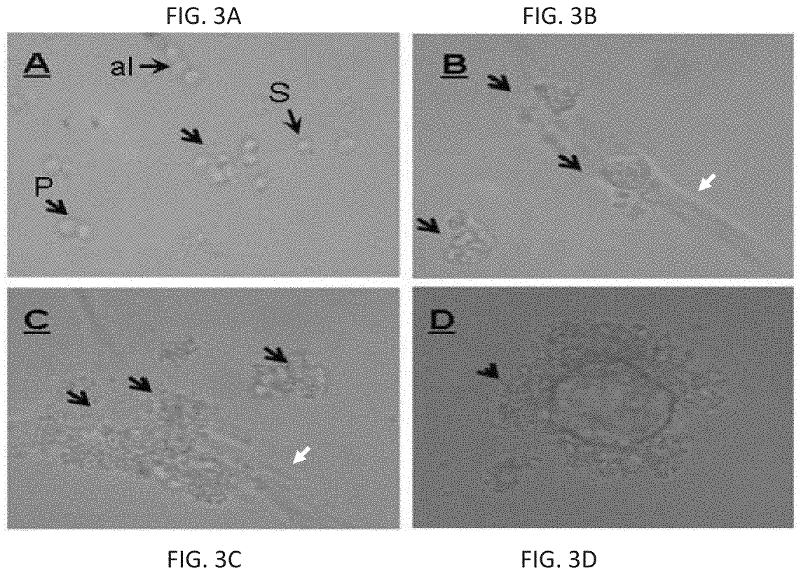

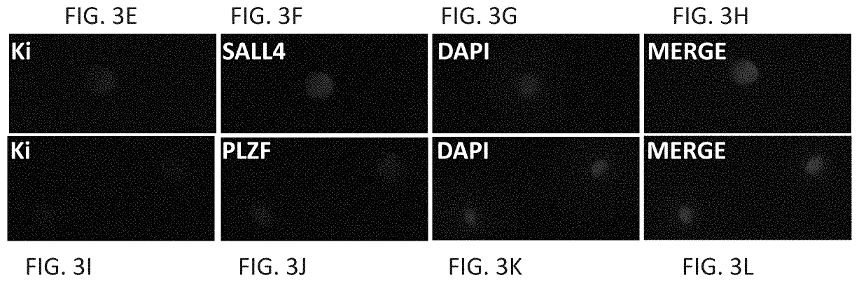

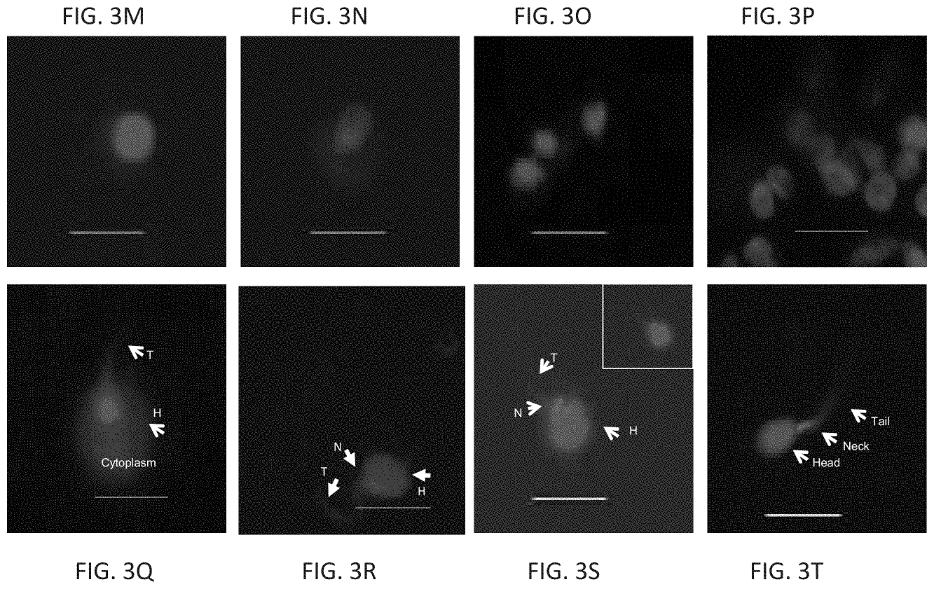

This study is the first to show a successful in vitro induction of meiotic and postmeiotic stages from spermatogonial cells harvested from some chemotherapy-treated prepubertal cancer patients, as shown by the development in culture of meiotic and postmeiotic germ cell types, and furthermore, the generation of cells with sperm-like morphology. In addition, without being bound by any theory, the results clearly show that biologically active SSCs (based on the presence/expression of several premeiotic markers before culture and their subsequent proliferation and development in the 3D culture to meiotic and postmeiotic stages) were present in the testes of prepubertal cancer patients even after chemotherapy treatments. It should be emphasized that even though the present inventor found bioactive SSCs in chemotherapy-treated patients, for some patients, there is a very high risk of no residual SSCs. Without being bound by any theory, this is dependent on type, dosages, and combinations of drugs/radiation and length of period of treatment. The developed cells from fresh and cryopreserved testicular tissues behaved similarly in MCS. Therefore, it is recommended to seek the advice of an oncologist regarding the need for cryopreservation of testicular biopsy before starting the chemotherapy/radiotherapy treatments. In one biopsy (Patient No. 4), the isolated cells only expressed PLZF, CREM-1 and BOULE before culture, and in another biopsy (Patient No. 2), the isolated cells did not stain for any of the examined spermatogenic markers before culture but stained in the tissue (before cell isolation) for PLZF, OCT4, CD9 and α6-Integrin, VASA, and c-KIT (Table 3). Also, patient No. 6 showed a Johnsen score of 5 by histological evaluation of his testicular biopsy (contained spermatocytes) but did not express meiotic markers in his isolated testicular cells. This could be related to the loss of cells following isolation procedure (some of the remaining cells are undetectable for some markers). These results show that most of the biopsies (6/8 patients) showed (before culture) the presence/expression of more than three premeiotic marker (3-7 markers) and only two patients who were older (10 and 13 years old) (Patient Nos. 4 and 5) expressed the presence/expression of only one marker (Table 3). The expression of only one premeiotic marker in testicular biopsy of patients Patient Nos. 4 and 5 could be related to the fact that these patients are close to puberty in age (10 and 13 years old, respectively), in which spermatogonial cells are more active in proliferation, which make them more sensitive to chemotherapy treatments. This could also be related to type, dosages, and combinations of drugs/radiation and length of treatment (31 and 29 months, respectively). Nurmio et al. 2009 (Nurmio M, “Effect of childhood acute lymphoblastic leukemia therapy on spermatogonia populations and future fertility”. J Clin Endocrinol Metab 94:2119-2122) demonstrated the presence of some markers of spermatogonial cells (four markers were examined) in testicular biopsies of some chemotherapy-treated prepubertal cancer patients. However, these markers were not detected in testicular biopsies of those patients who were treated with cyclophosphamide. In contrast, the results presented herein showed the presence of biologically active spermatogonial cells in cyclophosphamide-ALL treated patients. Furthermore, Poganitsch-Korhonen et al. 2017 (“Decreased spermatogonial quantity in prepubertal boys with leukaemia treated with alkylating agents”. Leukemia 7; 1-4) demonstrated that the quantity of spermatogonial cells (according to histology/cross section) decreased with treatment with alkylating agents. However, the results presented herein show that the spermatogonial cells, which were found in biopsies of some patients, were biologically active as they could proliferate and differentiate to different stages of spermatogenesis in MCS in vitro. The present inventor grew human spermatogonial cells in MCS, in similar conditions (temperature; 37° C.) used in a previous study of the present inventor with mouse systems [Abu et al. 2012], which showed similar results of development of spermatogenesis as compared to other cultured systems under 35° C. [Stukenborg J-B et al., 2009; Stukenborg J-B et al., 2008]. The similar effect of the different temperature on the development of spermatogenesis in vitro (which is in contrast to in vivo effects) could be related to a different microenvironment present in the in vitro conditions including the type of cells, proteins, constant conditions (flow/diffusion) and methylcellulose (not the normal extracellular matrices and 3D of the tubule) compared to in vivo conditions. The inability of isolated spermatogonial cells from two biopsies from ALL-chemotherapy-treated patients (patient Nos. 2, and 4) to proliferate and differentiate in MCS could be related to the quality and/or quantity of these cells and/or to the activity of the supporting cells present in the culture and originate from the biopsies of the patients (Table 3). On the other hand, isolated spermatogonial cells from the other three patients who received chemotherapy (Patient Nos. 3, 5, and 6), two out of whom even received cyclophosphamide (Patient Nos. 3, and 5), could proliferate and/or differentiate to meiotic and/or postmeiotic cells with no association to the type of disease or the kind of chemotherapy protocol (Table 3). Without being bound by any theory, these results may support the suggestion that the development of spermatogonial cells in MCS could be related to the quality of spermatogonial cells and to the activity of the supporting cells after chemotherapy treatment. On the other hand, without being bound by any theory the differences in timing of maturation in vitro for cells isolated from testicular biopsies of patients Nos. 3 and 6, even though they are of similar age (6 and 7 years old) could be due to the type of the disease (ALL and MD, respectively) and treatments they received. They even expressed different cell markers before culture. Also, it should be noted that isolated spermatogonial cells from patient No. 3 (ALL-chemotherapy treated including cyclophosphamide) developed in MCS meiotic (boule- and acrosin round positive cells), postmeiotic cells (acrosin-positive elongated cells) and even cells with sperm-like morphology, as detected by MitoTracker staining (FIG. 3M-3T). Some of the generated sperm-like cells were positively stained to acrosin and showed nucleus similar to that of normal sperm. However, other sperm-like cells showed nucleus larger than that of normal sperm. Without being bound by any theory these results may indicate that some of the generated sperm-like cells in MCS may be morphologically normal, while others are still premature sperm. These developed sperm-like cells in MCS are similar to those described in stages 8-12 of spermatid development in the human seminiferous epithelium VIII-XII stages [Muciaccia B, et al., 2013. “Novel stage classification of human spermatogenesis based on acrosome development”. Biol Reprod. 89:1-10]. The different morphology of sperm-like cells developed in the culture system compared to the morphology of the sperm from the “positive control,” could be related to either the degree of development (stage of development) of the sperm-like cells and/or to the culture conditions that may affect the morphology of the developed sperm. Thus, without being bound by any theory, these results may suggest the ability of spermatogonial cells from some ALL-cancer patients (even after chemotherapy treatment) to achieve almost complete spermatogenesis under certain in vitro conditions such as MCS. The expression of boule and protamine (RNA but not protein; immunofluorescence staining was undetectable) in isolated cells of this patient before culture may indicate the presence of cells from the early stages of meiosis that express these markers. Indeed, recently it was shown that protamine could be expressed from the pachytene stage of meiosis; however, the protein is expressed only at the spermiogenesis stage [da Cruz I, et al., 2016, BMC Genomics 17:294-313].

As shown in the Examples section below. in some cases, the expression (RNA) and the translation (protein—stained by IF) are not in parallel. Without being bound by any theory, when protein was detected but RNA did not, this could be related to regulation of RNA expression or its stability. However, without being bound by any theory, when RNA was detected but protein was not, this could be related to translational regulation. In addition, either protein levels and/or RNA expression could be related to the stage of cell development [da Cruz I, et al. 2016. “Transcriptome analysis of highly purified mouse spermatogenic cell populations: gene expression signatures switch from meiotic-to postmeiotic-related processes at pachytene stage”. BMC Genomics 17:294-313].

The in vitro culture system used according to the method of some embodiments of the invention to induce spermatogenesis was composed of MCS (a 3D system that mimics the in vivo conditions of the seminiferous tubules). In addition to the 3D conditions, different growth factors (GDNF, LIF, FGF, EGF), StemPro media and KSR were present in the MCS. These factors (GDNF, LIF, FGF, EGF) induced proliferation of mouse and human spermatogonial cells [Wu X, et al. 2009, Proc. Nat. Acad. Sci 106: 21672-21677; Sadri-Ardekani H, et al., 2011, J Am Med Asso 305: 2416-2418; and Kanatsu-Shinohara M, et al., 2003, Biol Reprod 69: 612-616]. In addition, Sertoli cells that produce functional factors, which are involved in induction proliferation and differentiation of spermatogonial cells were present in the culture. Also, peritubular and Leydig cells that may support the microenvironment of spermatogonial cell development in vivo were cultured in the culture system of some embodiments of the invention.

Without being bound by any theory, it is suggested that the 3D culture conditions (provided by MCS) and growth factors, in addition to the somatic cells present in the biopsies that remain in the culture, provide a microenvironment that supports and enables the development of spermatogenesis including, in one case, the generation of sperm-like cells. The conditions of the in-vitro culture are not yet optimized, and the type and quality of cells from the biopsies may also vary from one case to another. Furthermore, and without being bound by any theory, it is possible that the reasons for not observing all the postmeiotic markers examined in the same culture could be related to different stability of these markers in vitro, or the possibility that different markers of the postmeiotic stage are not expressed in the same time point and, thus, the number of cells expressing one marker could possibly not be in another marker. Since the in vitro culture of human spermatogonial cells from prepubertal cancer patient boys is new in in the MCS system, the present inventor has preferred examining the development of these cells from fresh biopsies rather than from frozen/thawed material. Once a fresh successful culture system is established, the frozen/thawed material can be further used. Using animal models, it was shown that cryopreserved spermatogonial cells and/or isolated spermatogonial cells from cryopreserved testicular tissue are still actively able to develop spermatogenesis in vivo [Giudice M C, et al. 2017. “Update on fertility restoration from prepubertal spermatogonial stem cells: How far are we from clinical practice?” Stem Cell Res 21: 171-77]. Additional studies can be performed to compare the efficiency of proliferation and differentiation of germ cells and the activity and viability of the supporting cells from cryopreserved and fresh biopsies in MCS. It is valuable to validate the efficiency of MCS for possible future use in the clinic.

The results presented herein can be considered as a proof of concept for the probability of induction of human spermatogonial cells from prepubertal cancer patients to develop almost last postmeiotic stages under specific in vitro conditions.

Without being bound by any theory, these results may encourage future therapeutic strategies using novel technologies (such as in vitro maturation or others) that may induce spermatogonial cells to generate round spermatids and/or sperm. Recently, it was shown that injection of human round spermatids to oocytes led to development of embryos and even to birth of newborns [Tanaka A, et al. 2015. “Fourteen babies born after round spermatid injection into human oocytes”. Proc Nat Acad Sci 112: 14629-14634]. The results presented herein can be used to induce the development of more meiotic and postmeiotic cells including the generation of normal and fertile sperm. The results presented herein show the presence of biologically active SSCs in testicular biopsies of chemotherapy-treated patients. Therefore, it is important to suggest that these survival cells may also develop in vivo and recover spermatogenesis in the cured patient after puberty, and the generated sperm could be used by assisted reproductive techniques to fertilize oocytes. It is important to emphasize the possible DNA damage and apoptotic triggered by the chemotherapy of SSCs, as well as the supporting somatic cells in the testis. Therefore, without being bound by any theory, it is recommended to cryopreserve testicular tissue from prepubertal cancer patient boys before chemotherapy/radiotherapy, and cryopreservation of testicular biopsy after chemotherapy/radiotherapy is an alternative approach for prepubertal cancer patient boys who have undergone cancer treatment without collection of testicular biopsy for fertility preservation. Thus, it is crucial to examine the epigenetic and DNA content of the cells before and after in vitro culture. Should this system be further validated and improved for the production of fertilization competent gametes, then it will circumvent the problem of fertility preservation of prepubertal cancer male patients who are receiving aggressive chemotherapy and/or radiation and still have some SSCs in their testes. This therapeutic approach will prevent the risk of reintroducing cancer cells into survivors by auto-transplanting testicular tissue/cell technologies. Furthermore, this technology may assist non-obstructive azoospermic patients in whom no sperm has been found in their testicular biopsies.

The results presented herein encourage the approach of cryopreserving testicular biopsies from prepubertal cancer patients that still contain biologically active spermatogonial cells to be used in future developed fertility therapeutic strategies (in vitro or in vivo).

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Methods

Human Testis Material from Prepubertal Patients—

Institution and national ethical committees approved the study. All patients' parents (or guardians) signed informed consent the fertility preservation procedure and to use part of their testicular biopsy for research. Testicular biopsies were obtained from 7 prepubertal cancer patients' boys (6-13 years old) with recurrent acute lymphoblastic leukemia (ALL) (n=4), medulloblastoma of Cerebellum (n=1), rhabdomyosarcoma (n=1), and acute promyelocytic leukemia (APML) (n=1) who are already treated with chemotherapy and additional patient which was used as a control for the chemotherapy-treated patient and was diagnosed as beta-thalassemia major, required frequent regular blood transfusions and was never treated with chemotherapy prior to the testicular biopsy.

All patients were scheduled for aggressive chemotherapy before bone marrow transplantation (BMT). According to the size of the biopsy, some of them were divided to be used for histological evaluation (fixation with 4% paraformaldehyde and embedding in paraffin) and/or for RNA extraction (stored at −70° C.), while others were used only for cell isolation to be cultured in vitro in MCS.

All patients were scheduled for aggressive chemotherapy before bone marrow transplantation (BMT). Tanner stage of development was evaluated for all patients, but the Johnsen score was performed on only three patients for whom there was histology for their testicular biopsies (Patients Nos. 2, 3 and 6). The present inventor also measured the diameters of the seminiferous tubules of these patients (presented as average of 35 STs±SD). Uro-genital history of the patients was not remarkable.

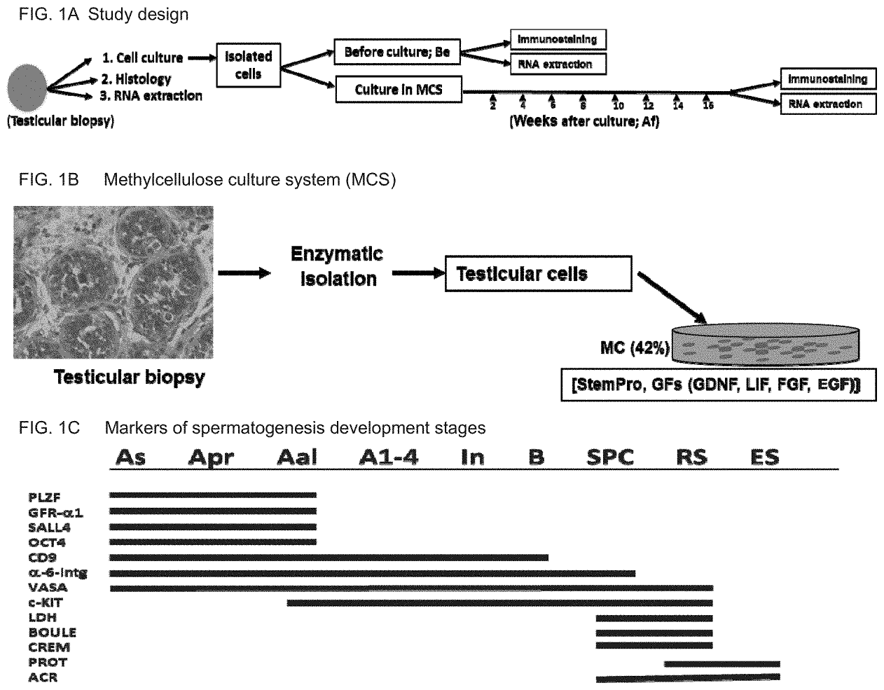

During the surgery, a single biopsy from one testis was taken in accordance with the approval of the ethical committee. Due to the small size of the biopsies used in the present study (around 3 mm³), most of the biopsies were used (as first priority) for in-vitro culture in MCS. In three biopsies, which were larger than 3 mm³, an additional part was used for histological evaluation (fixation with Bouin's solution and embedding in paraffin) and/or for RNA extraction (stored at −70° C.) (see study design in FIG. 1A).

Testicular Cell Isolation and Culture from Prepubertal Patients—

Seven of the biopsies were immediately transferred in Dulbecco's phosphate buffer saline (DPBS) in ice from the operating theater to the present inventor' lab for cell isolation.

Cryopreservation: One biopsy from a cancer patient male (“patient number 8”) was cryopreserved using cryoprotectant for 10 minutes in room temperature and thereafter stored in liquid nitrogen for 11 months. Briefly, for cryopreservation, after washing with PBS to remove residual blood, the biopsy was divided to around 3 mm³ parts and cryopreserved in 1.8 cryovials that contained 1.5 ml cryoprotectant media composed of 5% DMSO, 10% human serum albumin (HAS) and 3.5% sucrose diluted in HBSS. The cooling rate was 0.5° C./minute, with holding at 0° C. for 9 minutes, followed by a cooling rate of 0.5° C./minute, until −8° C. with a holding of 5 minutes at this temperature. Thereafter, seeding was manually performed. After 15 minutes holding at −8° C., the vials were frozen to −40° C. at a rate of 0.5° C./min. The vials were then frozen to −80° C. at a rate of 0.7° C./min and then transferred to liquid nitrogen. The cryopreserved biopsy was thawed in room temperature (RT) and centrifuged (for washing) in the presence of Minimum Essential Media (MEM) (Biological Industries).

Part of the biopsy from three patients (Patients Nos. 2, 3, and 6) were bouin-fixed and paraffin-embedded to be used for histological analysis by hematoxylin and eosin (HE) staining or immunostaining for different markers of spermatogenesis.

The fixation was performed immediately following the surgery of patients Nos. 2 and 6, and after thawing the cryopreserved biopsy of patient No. 3.

Biopsies were cut into small pieces of about 1 mm or about 2 mm, and were subjected to collagenase type V (2 mg/ml, e.g., Sigma, St. Louis, Mo., USA), DNAse (8 μg/ml, e.g., Sigma) and hyelorunidase (2 mg/ml, e.g., Sigma) in a total volume of 4 ml for 20 minutes in 32° C. water bath shaker. Cells were precipitated by centrifugation (300×g, 10 minutes) and suspended with 4 ml of Tryple Select (Invitrogen, Gibco, Denmark) for 10 minutes in a shaking water bath at 32° C. Cells suspension was centrifuged (300×g, 10 minutes) and the precipitated cells were suspended in 200 μl Roswell Park Memorial Institute (RPMI) medium (e.g., from Biological Industries, Beit Haemek, Israel) and counted.

Culturing in Methylcellulose (MC) Culture Systems—

Prior to culturing in the methylcellulose culture systems (MCS) the cells were cultured in a 24-well plate (uncotated 24-well plate) at a seeding concentration of 2×10⁴ cells/well/500 μl in MEM medium which contained sodium bicarbonate 7.5%, L-glutamine 200 mM, non-essential amino acids 1%, penicillin/streptomycin and gentamicin 10 mg/ml, and incubated over 2 nights at 37° C., 5% CO2.

The nonadherent cells were collected and cultured (2-5×10⁴ cells/well/500 μl; or at a concentration of 4-5×10⁴ cells/well/500 μl) in methylcellulose (42%; R&D, Minneapolis, USA) [as a three-dimension (3D) culture system]. The cells were diluted in 58% of media composed of 33% StemPro-34 medium and 25% KSR (knock-out serum replacement) (Gibco, USA) enriched with different factors and reagents such as human rEGF (recombinant epidermal growth factor) (20 ng/ml) (e.g., from Biolegend, CA, USA), human rGDNF (glial cell line derived nerve growth factor) (10 ng/ml) (e.g., from Biolegend), human rLIF (leukemia inhibitory factor) (10 ng/ml) (e.g., from Biolegend) and human r-bFGF (basic fibroblast growth factor) (10 ng/ml) (e.g., from Biolegend). Media containing isolated cells (58% final dilution in the well) were mixed with MC (42% final dilution in the well) and were cultured in the wells. Cells were cultured for 5-15 weeks in CO2 incubator at 37° C.

Alternatively, the cells were diluted in a medium which contained 33% StemPro-34 medium (Gibco, e.g., from USA and the StemPro supplement (2.6%; e.g., from Gibco), insulin (25 μg/ml; e.g., from Gibco), transferrin (100 μg/ml; e.g., from Gibco), putrescin (60 μg/ml; Gibco), sodium selenite (30 nM; e.g., from Gibco), D-glucose (6 mg/ml; e.g., from Sigma), pyruvic acid (30□ μg/ml; e.g., from Sigma), bovine serum albumin (BSA) (5 mg/ml; Millpore, Illkirch, France), L-glutamine (2 mM; e.g., from Biological Industries), 2-mercaptoethanol (0.5 μM; e.g., from Gibco), MEM vitamin solution (10 μl/ml; e.g., from Gibco, UK), MEM non-essential amino acid solution (10 μl/ml; e.g., from Gibco, UK), ascorbic acid (100 μM; e.g., from Sigma, China), d-biotin (10 μg/ml; e.g., from Sigma), 1% KSR, 0.5% (e.g., from Gibco, UK), Pen/Strep (e.g., from Biological Industries), enriched with different factors and reagents such as human rEGF (20 ng/ml; Biolegend, CA, USA), human rGDNF (10 ng/ml; Biolegend), human rLIF (10 ng/ml; Biolegend) and human r-bFGF (10 ng/ml; Biolegend). Cells were cultured for 1-16 weeks.

Every 1-2 weeks (according to the growth and morphology of the cells) the present inventor added 50 μl/well of fresh concentrated (×10) enriched StemPro-34 medium to the cell cultures.

Every 10 days to 2 weeks the present inventor has evaluated the development of the cells in MCS under the microscope [according to the growth quality/viability (around 10% of the cells with apoptotic vacuoles in their cytoplasm) and morphology of the developed colonies] and added 50 μl/well of fresh concentrated (×10) enriched StemPro-34 medium (containing all the growth factors used in the primary culture) to the cell cultures to be followed up after additional 1-2 weeks (see design of the study in FIG. 1A). The present inventor tried to grow the cells in the culture as much as possible to be closer to the physiological timing of development of human spermatogenesis (around 3 months). At the end of the incubation period in MCS, the present inventor added 0.5 ml PBS to the culture wells that contained 0.5 ml MC mix by pipetting and collected the suspension to 15 ml tubes. The tubes were centrifuged in 1600 RPM for 10 minutes. Most of the volume was removed and the remainder around 100 μl from the bottom of the tube was collected. This volume which contained the cells was smeared on a slide and/or collected and kept at −70° C. to be used for RNA analyses (according to the number of colonies developed in the culture) In case there were more than 10-15 small colonies, or 5 medium or large colonies in the well, the cells were utilized for both IF and RNA analyses. Otherwise, the priority was for IF analysis.

Human Adult Testis Material from adult patients Institutional ethical committees approved the study. All patients signed informed consent to use their testicular biopsy for research. Testicular specimens were obtained from 61 azoospermic patients who were referred to the assisted reproductive technique in the IVF program according to the absence of sperm in their ejaculate after centrifugation and meticulous search of sperm cells. The specimens were obtained either by testicular sperm extraction (TESE; n=49) or testicular fine needle aspiration (TEFNA; n=12). The age of the patients ranged from 21-57 years. TESE specimens were histologically identified according to the different groups of NOA, while TEFNA specimens were not subject to histological evaluation. In addition, testicular specimens from Klinefelter syndrome patients were used (Kf; n=5). The testicular specimens were divided in the laboratory (after thorough search for sperm) to specimens that contained sperm [(+) sperm] or specimens without sperm [(−) sperm]. Specimens were divided for histological evaluation and/or for RNA extraction, while others were also used for in vitro culture.

Testicular Cell Isolation and Culture from Adult Patients

After an overnight in sperm wash media containing 30% human serum albumin at room temperature (RT), TESE specimen were re-searched for sperm and then collected into the same media and transferred to the present inventor' lab, while TEFNA specimens were transferred to Modified Human Tubal Fluid (HTF) media containing 15% serum protein substitutes and transferred to the present inventor' lab after two days at RT. For cell isolation, biopsies were cut into small pieces of ˜2 mm, and subjected to collagenase type V (2 mg/ml) (Sigma, St. Louis, Mo., USA), DNAse (8 μg/ml) (Sigma) and hyelorunidase (2 mg/ml) (Sigma) in a total volume of 4 ml for 20 minutes in a 32° C. water bath shaker. Cells were precipitated by centrifugation (300×g, 10 minutes) and suspended with 4 ml of Tryple Select (Gibco, Denmark) for 10 minutes in a shaking water bath at 32° C. Cell suspension was centrifuged (300×g, 10 minutes) and the precipitated cells were suspended in 200 μl Roswell Park Memorial Institute (RPMI) (Biological Industries, Beit Haemek, Israel) and counted. Cells were cultured (2×10⁴ cells/well/500 μl) in Minimum Essential Medi (MEM) (Biological Industries) which contained sodium bicarbonate (Gibco, UK), 7.5%, L-glutamine (Biological industries) 200 mM, non-essential amino acids (Gibco, UK) 1%, penicillin/streptomycin (Biological Industries) and gentamicin (Biological Industries) (10 mg/ml), and incubated for 2 nights in 24 well plates at 37° C., 5% CO2. The nonadherent cells were collected and cultured (2×10⁴ cells/well/500 μl) in methylcellulose (R&D, Minneapolis, USA) (42%) [as a three-dimension (3D) culture system], which contained 33% StemPro-34 medium (Gibco, USA) enriched with: StemPro supplement (2.6%) (Gibco), insulin (25 μg/ml) (Gibco), transferrin (100 μg/ml) (Gibco), putrescin (60 μg/ml) (Gibco), sodium selenite (30 nM) (Gibco), D-glucose (6 mg/ml) (Sigma), pyruvic acid (30 μg/ml) (Sigma), BSA (Millpore, Illkirch, France) (5 mg/ml), L-glutamine (2 mM) (Biological Industries), 2-mercaptoethanol (0.5 μM) (Gibco), MEM vitamin solution (10 μl/ml) (Gibco, UK), MEM non-essential amino acid solution (10 μl/ml) (Gibco, UK), ascorbic acid (100 μM) (Sigma, China), d-biotin (10 μg/ml) (Sigma), 1% KSR (Gibco, UK), 0.5% Pen/Strep (Biological Industries), human rEGF (20 ng/ml) (Biolegend, CA, USA), human rGDNF (10 ng/ml) (Biolegend), human rLIF (10 ng/ml) (Biolegend) and human r-bFGF (10 ng/ml) (Biolegend). Cells were cultured for 1-16 weeks. Every 1-2 weeks (according to the growth and morphology of the cells), we added 50 μl/well of fresh concentrated (×10) enriched StemPro-34 medium to the cell cultures.

Immunofluorescence Staining—

To determine a specific germ cell population distinctive to each spermatogenic phase in the testicular sections or in isolated testicular cells before and after in vitro culture in MCS, immunofluorescence staining was performed to markers known to be specific for the different stages of spermatogenesis (20,21) (FIG. 1C).

Testicular Tissues Staining—

Testicular biopsies were fixed in 4% paraformadehyde or in Bouin's solution (Kaltek, Italy) and paraffin-embedded. Sections of 5 μm were placed on SUPERFROST® PLUS slides (Thermo, Braunschweig, Germany) for immunofluorescence staining of spermatogenic markers. Deparaffinated section slides were treated with xylene and ethanol for 20 minutes. After washing with phosphate buffer solution (PBS), antigen retrieval of the sections was performed in heated 36% urea solution (Millpore) at warm microwave degree (900 Watts) for 5 minutes (twice). After washing, nonspecific adhesions sites in the tissues and cells were blocked by 5% fetal calf serum (FCS) (e.g., from Biological Industries) for 30 minutes at room temperature. After removing the blocking buffer, the first antibodies were added. The first antibodies included polyclonal rabbit anti-human VASA (Santa Cruz, Calif., USA; 1:1000), Polyclonal rabbit anti-human CD9 (Abcam, Cambridge, UK; 1:200), Polyclonal goat anti-human OCT4 (Santa Cruz; 1:200), Polyclonal rabbit anti-human α-6-INTEGRIN (Santa Cruz; 1:200), Polyclonal goat anti-human GFR-α (R&D, MN, USA; 1:50), monoclonal mouse anti-human PLZF (Santa Cruz; 1:100), Polyclonal rabbit anti-human c-KIT (Dako, CA, USA; 1:200), Polyclonal goat anti-mouse vimentin (Santa Cruz; 1:50) and Polyclonal rabbit anti-mouse GDNF (Santa Cruz; 1:100). After overnight incubation 4° C., the slides were washed and the specific secondary antibodies were added compatibly to the first antibodies for 40 minutes at room temperature. The secondary antibodies were Donkey anti-rabbit IgG (Cy3), Donkey anti-goat IgG (Cy3), and Goat anti-mouse IgG (Rhodamine red) Jackson Immuno Research (USA).

After washing, the slides dried and DAPI, which stains the nuclei in blue, was added to the tissues and the slides were stuck with cover slides. The negative control was incubated in blocking buffer instead of the first antibody. Slides were examined for staining using a fluorescence microscope (Nikon eclipse 50 i).

The specificity of the staining was also examined in testicular tissue using the relevant IgG isotype as negative control. The positive staining for the examined markers was performed on bouin-fixed adult human testis tissue embedded in paraffin. Slides were examined for staining using a fluorescence microscope (Nikon eclipse 50 I, Tokyo, Japan).

Immunostaining of Testicular Cells:

Isolated cells were fixed in cold methanol for 20 minutes. The immunostaining process was similar to that mentioned above for testicular tissue immunostaining after the stage of antigen retrieval. Following the removal of the blocking buffer, the first antibodies were added: Polyclonal rabbit anti-mouse SALL4 (Abcam, Cambridge, UK; 1:400), Polyclonal rabbit anti-human VASA (Santa Cruz 1:100), Polyclonal rabbit anti-human CD9 (Santa Cruz; 1:10), Polyclonal goat anti-human OCT4 (Santa Cruz; 1:50), Polyclonal rabbit anti-human α-6-INTEGRIN (Santa Cruz; 1:100), Polyclonal rabbit anti-human GFR-α □(Santa Cruz; 1:50), monoclonal mouse anti-human PLZF (Santa Cruz; 1:100), Polyclonal rabbit anti-human c-KIT (Santa Cruz; 1:50), Polyclonal rabbit anti-human BOULE (Santa Cruz; 1:25), Polyclonal rabbit anti-mouse CREM-1 (Santa Cruz; 1:30), Polyclonal goat anti-human LDH (Santa Cruz; 1:50), Polyclonal goat anti-mouse PROTAMINE (Santa Cruz; 1:20), Polyclonal rabbit anti-human ACROSIN (Santa Cruz; 1:10), monoclonal mouse anti-human Ki67 (Dianova GmbH, Germany; 1:200), monoclonal rabbit anti-human Ki67 (Cell marque; 1:200). After overnight incubation at 4° C., the slides were washed and treated as mentioned above for testicular tissue immunostaining. Since the antibodies are cross reactive with mouse, the positive staining for the examined markers was performed using isolated testicular cells from azoospermic patients with sperm in their biopsies and/or mouse testicular cells.

Counting of Stained Cells:

Slides were divided by PAP pen to be used for staining of a few markers. For each marker, about 30 cells were counted when the number of positive stained cells was dependent on the examined marker or patient and if the sample was from before or after culture. For example, in one case 5/32 VASA-positive stained cells, 6/23 PLZF-positive stained cells and 5/31 LDH-positive stained cells were counted.

MitoTracker Staining:

MitoTracker Green FM probes (Molecular Probes, Catalogue number 7514, Invitrogen), which stain mitochondria, were used to identify developed sperm in MCS, according to the supplier's protocol. Briefly, 200 nM MitoTracker was added to collected cells from MCS (in an Eppendorf tube), and incubated at 37° C. in CO2 incubator for 30 minutes. Thereafter, the tubes were centrifuged at 1600 RPM for 5 minutes. At the end of the centrifugation, the supernatant was removed, and the pellet was diluted with 50 μl PBS. DAPI was added to the tube (5 μl) to the tube, and the suspension was smeared on a slide. Identification of sperm-like cells was performed immediately using a fluorescence microscope (Nikon eclipse 50 I, Tokyo, Japan).

Gene Expression—

In some testicular tissues and cells, the gene expression of the spermatogenic markers were also examined (according to the amount of tissue/cells remained after cell isolation or at the end of the culture in MCS) using specific primers for each one.

Isolated Cells:

Enzymatically-isolated testicular cells and developed cells from in vitro cultures were mixed with 200 μl of lysis buffer (Dynabeads Kit; Dynal Biotech, Oslo, Norway).

The lysates were frozen at −80° C. for later RNA extraction using a Dynabeads kit (Invitrogen, Lithuania).

The cDNA synthesis was performed according to M-MLV Reverse Transcriptase protocol (Invitrogen, USA) using random hexamers, and PCR was performed using specific primers for each examined spermatogenesis markers.

Testicular Tissue—

Total RNA was extracted from testicular tissues by Trizol reagent according to the manufacturer's instructions (Sigma, St. louis, Mo.). 0.7 μg of each testicular biopsy's total RNA was transformed into cDNA by reverse transcription reaction performed by RevertAid First Strand cDNA Synthesis Kit (Fermentas, Burlington, Canada). 1.5 μl of the cDNA product was reinforced by quantitative real time—polymerase chain reaction (Q-RT-PCR).

The evaluation of the pre-meiotic spermatogonial genes OCT-4 (forward: AATTTGCCAAGCTCCTGAAG (SEQ ID NO:1); reverse: CGTTTGGCTGAATACCTTCC (SEQ ID NO:2); product size, 337 base pairs [bp]), SALL-4 (forward: TCCCAAACACCAGITTTCCTC (SEQ ID NO:3); reverse: TGTGTCTGCATTGCTCCTTC (SEQ ID NO:4); product size, 90 bp), a-6-integrin (forward: TTGTTTCGTAACACAGCATTG (SEQ ID NO:5); reverse: GGCACTAGTATCTTTGGCTGA (SEQ ID NO:6); product size, 146 bp), CD-9 (forward: CCTACAACAAGCTGAAAACCA (SEQ ID NO:7); reverse: GGATAGCACAGCACAAGATCA (SEQ ID NO:8); product size, 282 bp), GFRa1 (forward: AGCAGGGTCTGAGAATGAAAT (SEQ ID NO:9); reverse: GCCATTGATTTTGTGGTTATG (SEQ ID NO:10); product size, 171 bp) and c-KIT (forward: TTCTACAAGATGATCAAGGAAGG (SEQ ID NO:11); reverse: AGAATTGATCCGCACAGAAT (SEQ ID NO:12); product size, 243 bp), the meiotic gene CREM (forward: ACGAGGTCCGCTACGTAAAT (SEQ ID NO:13); reverse: GGCTCTCCAGACATTTTACATATT (SEQ ID NO:14); product size, 249 bp) and the post-meiotic gene PROTAMINE (forward: AAAGAAGTCGCAGACGAAGGA (SEQ ID NO:15); reverse: TATTGGATGGTGGCATTTTCA (SEQ ID NO:16); product size, 193 bp) in testicular biopsies was performed. The relative concentration of these genes expression was estimated in duplicates with Eco™ Real-time PCR System (Illumina) using SYBR Fast Universal Readymix kit (KapaBiosystem, Boston, USA). The analysis of the relative gene expression was presented according to the comparative CT method: 2-ACT, which exhibits the evaluated gene expression fold regarding the calibrator PPIA gene (forward: TATCCTAGAGGTGGCGGATTT (SEQ ID NO:18); reverse: GAATGGTATCACCCAGGGAAT (SEQ ID NO:19); product size, 150 bp).

Extraction of RNA from Isolated Cells—

Enzymatically isolated testicular cells and developed cells after in vitro culture were centrifuged at 13,000 rpm for 5 minutes. The sediment was mixed with 200 μl of lysis buffer which is supplied with the Dynabeads Kit. The lysates were frozen at −80° C. till the RNA extraction stage. RNA was extracted from testicular germ cells using Dynabeads kit which contains magnetic beads binding poly-dT molecules that bind to m-RNA poly-A.

RT-PCR from Isolated Cells—

The cDNA synthesis was according to M-MLV Reverse Transcriptase protocol (invitrogen) using random hexamers, and PCR was carried out with the spermatogenic markers specific primers described above, in addition to VASA (forward: TGG AAA CAG AGA TGC TGG TG (SEQ ID NO:20); reverse: CCT CTG TYC CGT GTT GGA TT (SEQ ID NO:21); product size, 183 bp), PLZF (forward: AAGGCTGCAGTGGACA (SEQ ID NO:22); reverse: CTGCATCATCATCTCCGTCTT (SEQ ID NO:23); product size, 144 bp), Boule (forward: CCATTTATCAGCAACCTGCAT (SEQ ID NO:24); reverse: GTGCAATTTCCACTGGTTGAT (SEQ ID NO:25); product size, 153 bp), LDH (forward: GGAACGGATTCAGATAAGGAA (SEQ ID NO:26); reverse: TTCACAACATCTGAGACACCA (SEQ ID NO:27); product size, 260 bp) and Acrosin (forward: CCGGCTGGGGATATATAGAA (SEQ ID NO:28); reverse: ACCACATAGGCGCTTTCCTT (SEQ ID NO: 17); product size, 229 bp).

The PCR amplification reaction was occurred as: 3 minutes at 95° C., 30 cycles of 15 seconds at 95° C., 15 seconds at 60° C. (all the primers were designed with this specific annealing temperature), and 40 seconds at 72° C. The final elongation step was for 5 minutes at 72° C.

Table 1, hereinbelow, summarizes the clinical data related to the prepubertal patient boys.

TABLE 1
Summary of clinical data relate to prepubertal patient boys

| | | | | | | Time lapse | | |
|---|---|---|---|---|---|---|---|---|
| | | | | Duration | | between last | | |
| Patient | Age | | | of chemo | | chemo and | Johnsen's | Tanner |
| No. | (Y) | Diagnosis | Treatment history and accumulative dosage | treatment | | surgery | score | stage |
| 1 | 6 | Acute | Chemotherapy + ATRA | 3 | M | 5 Y | ND | I |
| | | Promyelocytic | [Etoposide, 450 mg; Idarubicin, 61 mg; Mitoxantrone, 20 | | | | | |
| | | Leukaemia (APML) | mg; Cytosar (3 months), 13400 mg; ATRA) 3 years), | | | | | |
| | | | Cytozar (IT)] | | | | | |
| 2 | 6 | Acute lymphoblastic | Chemotherapy (BFM ALL 2009 protocol) | 4 | M | 1 M | 4 | I |
| | | leukemia (ALL) | [Frednizon/Dexcorate (4 months), Vincristin (3 months), | | | | | |
| | | | 15.1 mg; Daunotubicin (3 months), 188.9 mg; | | | | | |
| | | | Aspargenase, 6297.2 mg; Cyclophosphamide (3 months), | | | | | |
| | | | 5 g; Ephosphomide, 5 g; Cytozar (3 months), 14.1 g; | | | | | |
| | | | Methotrexate, 12.6 g; Autophoside, 629.7 mg]. | | | | | |
| 3 | 7 | Acute lymphoblastic | Chemotherapy (BFM ALL 2009 protocol) | 30 | M | 1 M | 4 | I |
| | | leukemia (ALL) | (Daunorubicin, 60 mg; Vincristin, 12 mg; Ducsorubicin, | | | | | |
| | | | 120 mg; Methotrixane, 20 g; Cyclophosphamide, 3 g; L- | | | | | |
| | | | Asparginz, 120000 Units; Cytozar, 1800 mg; Lanbis, 840 | | | | | |
| | | | mg; Purinetol, 3080 mg; Ferdnizon, 1800 mg; | | | | | |
| | | | Dexamrthasone, 236 mg) | | | | | |
| 4 | 10 | Acute lymphoblastic | Chemotherapy (BFM ALL 2009 protocol) | 31 | M | 1 M | ND | II |
| | | leukemia (ALL) | [Deonorubicin (6 months), 240 mg; Eidrubicine, 24 mg; | | | | | |
| | | | Cyclophosphamide (6 months), 4 g; Vincrystine (7 | | | | | |
| | | | months), 20.5 mg; Cytarabine (20 months), 8400 mg; | | | | | |
| | | | Peg-Aspargenase (7 months), 5000 Units; Arabinase, | | | | | |
| | | | 200000 Units; Methotrixate (3 months), 21000 mg; | | | | | |
| | | | Parddenizane (7 months) | | | | | |
| 5 | 13 | Acute lymphoblastic | Chemotherapy (BFM ALL 2009 protocol) | 29 | M | 1 M | ND | III-IV |
| | | leukemia (ALL) | (Predinisone, 2340 mg; Vincistine, 15.6 mg; | | | | | |
| | | | Daunorabicine, 156 mg; L-Asparginase, 156000 Units; | | | | | |
| | | | Cycophosphamide, 3.9 g; Cytarabine, 2340 mg; | | | | | |
| | | | Mercaptopurine, 39650 mg; Methotrexate IV, 26 g; | | | | | |
| | | | Methotrexate PO, 2184 mg; Dexamethasone, 325 mg; | | | | | |
| | | | Doxorubicine, 156 mg; Thioguanine, 1092 mg) | | | | | |
| 6 | 6 | Medulloblastoma | Chemotherapy (Vincristine), Cranial-Spinal Radiation | Once | | 2 M | 5 | I |
| | | (MD) | (Gy 23.4), Vincristine, 1.5 mg. | | | | | |
| 7 | 9 | Rhabdomyosarcoma | Chemotherapy, Radiation | 10 | M | 8 M | ND | I |
| | | (recurrent) | (Vincristine, 1.5 mg; Actinomycin, 15 mg; Cytoxan (all | | | | | |
| | | | for 8 months), 25 mg; VP16, 500 mg; Ifosfamide, 10 g; | | | | | |
| | | | Doxorubicin) and auto stem cell transplantation | | | | | |
| | | | (Thiotepa, 720 mg; melphalan, 180 mg; Carboplatin, 2 g). | | | | | |

| | | | | | | | |
|---|---|---|---|---|---|---|---|
| 8 | 7 | Beta-Thalassemia | None | None | None | ND | I |
| | | Major (THA) | | | | | |

Table 1.

### Example 1

### In Vitro Maturation of Human Spermatogonia Obtained from Prepubertal Boys

Experimental Results

Testicular Biopsy—

Testicular biopsies were used from 8 prepubertal boys (6-13 years old); seven out of them after chemotherapy for cancer treatment prior to testicular biopsy, and one without prior chemotherapy treatment. All of them were assigned to aggressive chemotherapy post testicular biopsy. The effect of TNF-α was examined on testicular cells isolated from biopsies of azoospermic patients. Testicular biopsies before or after enzymatic digestion (isolated cells) were analyzed by immunofluorescence staining (IF) or by PCR analysis for spermatogenic markers. Cells were cultured in methylcellulose 3D in vitro culture system (MCS) in the presence of different growth factors. The cells were examined after 5-15 weeks in MCS.

The results demonstrate the presence (by IF and/or PCR) of some pre-meiotic markers before culture in 8 out of the 8 cases (oct4, vasa, plzf, sall4, gfra, cd9, a6-integrin, c-kit), some meiotic markers (crem-1, Idh, boule) in 6/8 of the cases and some post-meiotic markers (protamine or acrosin) in 2 out of 8 of the examined biopsies. The present inventor was able to culture cells out of four biopsies (50%). Cells from two biopsies (6 and 7 years old) that did not show meiotic markers before culture showed some of those markers after culture. Cells from one biopsy (6 years old) that did not show presence of post-meiotic markers before culture, showed only protamine after culture. A different biopsy from a 7 year old boy that expressed protamine (only by PCR but not stained by IF) but did not show acrosin (by PCR and IF) before culture, did not express protamine after culture, but showed acrosin (IF) and even very few sperm-like cells.

Histology and Immunofluorescence Staining of Spermatogenic Cells in Testicular Biopsies from Pre-Pubertal Cancer Patient Boys—

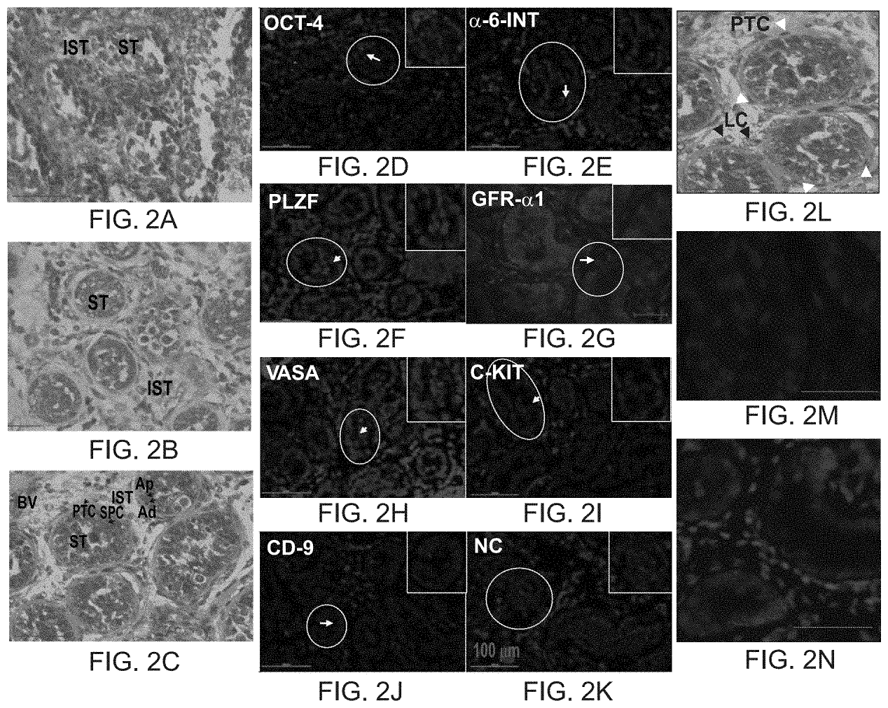

Histological sections of testicular biopsies from three pre-pubertal ALL cancer patient boys (FIG. 2A: Patient No. 2, FIG. 2B: Patient No. 3) and one MD patient (FIG. 2C: Patient No. 6) showed similar diameters (μm±SD; 133±11, 124±10 and 141±10, respectively) of their seminiferous tubules (STs). The presence of A dark (Ad) and A pale (Ap) spermatogonial cells was distinguished according to the intensity of staining of the nucleus by HE staining, and their close to the basal membrane of the ST. In two of the patients small seminiferous tubules (ST) with small lumen and Ad (Ad) and Aₚₐₗₑ (Ap) spermatogonial cells close to the basal membrane of the ST. Primary/secondary spermatocytes (SPC) are also recognized and distinguished according to their location inside the ST of some biopsies and the morphology of the nucleus (FIGS. 2B-C). In the periphery of the seminiferous tubule (outside the tubules) peritubular cells (PTC) are shown, and normal interstitial tissue (IST) which composed mainly of Leydig cells (LCs; FIGS. 2B-D), macrophages, blood (BV) and lymph vessels (FIGS. 2B-C) are also shown. However, the histology of the STs in patient No. 2 showed a wider lumen and impaired STs with a thin single layer of cells (FIG. 2A) compared to patients Nos. 3 and 6 (FIGS. 2B and 2C, respectively). Also, the IST was unorganized compared to the other two patients (FIG. 2A).

Immunofluorescence staining of testicular biopsies from pre-pubertal cancer patient boys (n=3, patient Nos. 2, 3, and 6) showed positive staining for the pre-meiotic markers VASA, PLZF, CD-9, α-6-INTEGRIN and C-KIT in all the 3 examined biopsies, whereas OCT-4 was stained in 2 of the 3 biopsies (Patients Nos. 2 and 3), and GFRα1 was stained only in one of the 3 biopsies (Patient No. 6; FIGS. 2D-K, and Tables 2 and 3, hereinbelow).

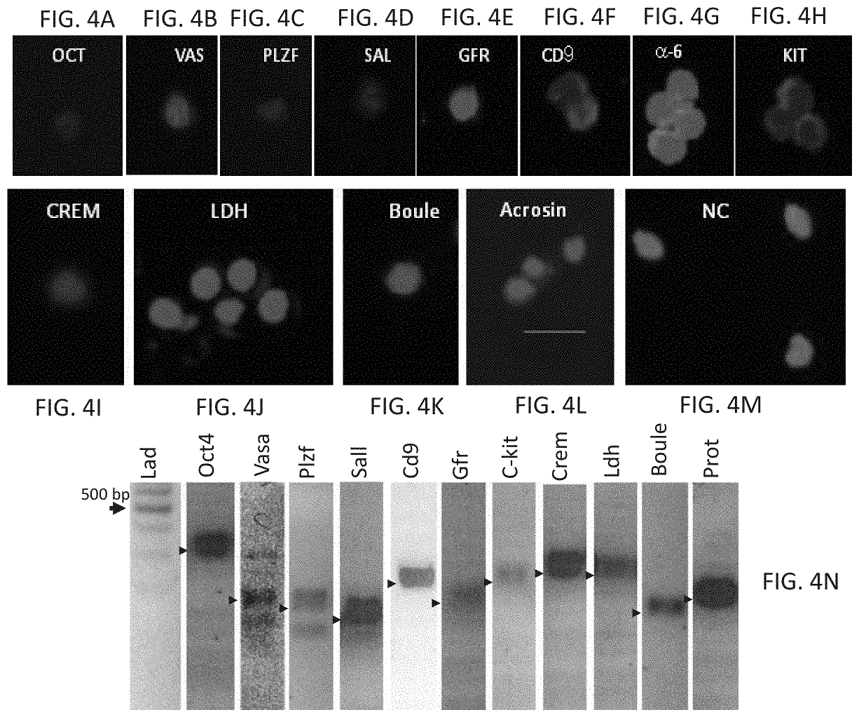

The present inventor has further examined sections by IF (FIGS. 4A-M) and/or RNA expression (FIG. 4N) for meiotic and postmeiotic markers, but those markers were undetectable. Immunostaining for Sertoli cells using specific antibodies to vimentin showed the presence of Sertoli cells in the seminiferous tubules of fixed tissue before enzymatic digestion (FIGS. 4A-M). These Sertoli cells were active to produce GDNF, as examined by a double staining for Sertoli cells (vimentin; red) and GDNF (green) (FIG. 4N). This is important in order to confirm that Sertoli cells (and not germ cells that also present in the tubules and known to produce GDNF) are functional/active and produce GDNF. These supporting cells are present in the culture and support the development of germ cells. The IF staining was performed on isolated cells from the biopsies of chemotherapy-treated patients and the control patient (untreated with chemotherapy prior to testicular biopsy; beta thalassemia major patient) (FIGS. 4A-M). The results are summarized in Table 2 and 3 below.

TABLE 2
Expression of spermatogenic markers in isolated cells from testicular biopsies of pre-pubertal cancer male patients before in vitro culture

| Stage | | Pre-meiotic | | | | | | | | | | | | | | | | | | | | | | | |
|---|---|---|---|---|---|---|---|---|---|---|---|---|---|---|---|---|---|---|---|---|---|---|---|---|---|
| Marker | | oct4 | | | vasa | | | plzf | | | sall4 | | | gfra | | | cd9 | | | a-6-int | | | ckit | | |
| Pt. No. | Age | R | IF | T-IF | R | IF | T-IF | R | IF | T-IF | R | IF | T-IF | R | IF | T-IF | R | IF | T-IF | R | IF | T-IF | R | IF | T-IF |

| | | | | | | | | | | | | | | | | | | | | | | | | |
|---|---|---|---|---|---|---|---|---|---|---|---|---|---|---|---|---|---|---|---|---|---|---|---|---|
| 1 | 6 | | | − | | | + | | | + | | | + | + | + | + | + | + | | | + | − | + | + |
| 2 | 6 | | | | | | | | | | | | + | + | | + | + | | − | + | | − | + | |
| 3 | 13 | | | | | | | | | | | | + | | | − | | | | | | − | | |
| 4 | 9 | + | | | | | | | | | + | − | − | + | | − | + | | | | | + | − | |
| 5 | 10 | − | − | | − | − | | + | − | | − | − | − | − | | − | − | | − | − | | − | − | |
| 6* | 7 | − | + | | − | + | | − | + | | − | − | − | − | | − | − | | − | − | | − | − | |
| 7 | 6 | | − | + | | − | + | | − | + | | − | | − | − | | − | + | | − | + | | − | + |
| 8 | 7 | + | − | + | + | + | + | + | − | + | + | − | − | − | − | − | + | + | − | − | + | + | − | + |

| | | | | | | | | |
|---|---|---|---|---|---|---|---|---|
| n/N | 4/6 | 4/5 | 5/5 | 2/5 | 4/8 | 5/8 | 4/6 | 5/8 |
| % | 67% | 80% | 100% | 40% | 50% | 63% | 67% | 63% |

Table 2: Expression of spermatogenic markers in isolated cells from testicular biopsies of pre-pubertal cancer male patients and a beta thalassemia major patient (*) before in vitro culture. “Pt.” = patient. “No.” = number; Isolated cells and/or testicular tissue (T) from biopsies of pre-pubertal cancer male patients (n = 8 and n = 3, respectively) were examined for pre-meiotic markers; (oct-4, plzf, vasa, gfra1, cd-9, a-6-integrin and c-kit) by immunofluorescence staining (IF) or by RT-PCR analysis (R) using specific primary antibodies or primers (respectively) for each marker. The results (+) or (−) indicate the presence or absence (respectively) of the marker are according to at least one of the analyses (IF and/or PCR) that were used. Empty Table's cells − indicate not examined. *- Indicate patient diagnosed with beta thalassemia major, n- Number of patients positively expressed the marker. N- Number of all the examined patients for the specific marker. n/N and (%)- The calculation of the fraction (or percentage) of samples examined at least in one methodology.

TABLE 3
Expression of spermatogenic markers in isolated cells from testicular
biopsies of pre-pubertal cancer male patients after in vitro culture

| Post- | | | | | | | | | | | | | Stage | | |
|---|---|---|---|---|---|---|---|---|---|---|---|---|---|---|---|
| meiotic | | Meiotic | | | Pre-meiotic | | | | | | | | Marker | | |
| acr | prot | boule | idh | crem | ckit | a-6-int | cd9 | gfra | sall4 | plzf | vasa | oct4 | BC/AC | Age (y) | Pt. No. |
| − | − | | − | − | + | | + | + | | | | | BC | 6 | 1 |
| − | + | | + | + | − | | + | + | | | | | AC (5 Ws) | | |
| − | − | | + | − | − | | − | + | | | | | BC | 13 | 3 |
| − | − | | − | − | + | | − | + | | | | | AC (8 Ws) | | |
| − | − | + | − | + | − | − | − | − | − | + | − | − | BC | 10 | 5 |
| − | − | − | − | | − | − | − | − | − | − | − | − | AC (8 Ws) | | |
| − | − | − | + | − | − | − | − | − | − | + | + | + | BC | 7 | 6 |
| − | − | + | − | − | − | + | + | − | + | + | + | + | AC (9 Ws) | | |
| − | − | − | − | − | − | − | − | − | − | − | − | − | BC | 6 | 7 |
| − | − | − | − | − | − | − | − | − | − | − | − | − | AC (11 Ws) | | |
| − | + | + | − | − | + | − | + | − | + | + | + | + | BC | 7 | 8 |
| + | − | + | | − | − | + | − | + | − | − | + | + | AC (15 Ws) | | |

Table 3: Expression of spermatogenic markers in developed cells/colonies in MCS from testicular cells of pre-pubertal cancer male patients. Cells isolated from testicular biopsies of pre-pubertal cancer male patients (n = 6) were cultured for number of weeks in vitro in MCS. Cells before and after culture in MCS were examined for the presence of spermatogenic markers by PCR analysis using specific primers or by immunofluorescence staining using specific antibodies for each spermatogenic marker: pre-meiotic (oct-4, plzf, vasa, gfra1, cd-9, a-6-integrin, sall4 and c-kit), meiotic (crem1, ldh and boule) and post-meiotic (protamine and acrosin), “Pt.” = patient. “No.” = number; “BC” = before culture; “AC” = after culture. Empty Table's cells = not examined, “Ws” = weeks.

TABLE 4
Summary of expression/presence of spermatogenic markers in developed cells/colonies in MCS
compared to before culture from testicular cells of pre-pubertal cancer patient boys

| | | | | Premeiotic markers | | | | | | | | | | | | | | | |
|---|---|---|---|---|---|---|---|---|---|---|---|---|---|---|---|---|---|---|---|
| Patients | | | | | | | | | | | | | | a-6- | | | | c- | |
| | Age | | Culture | PLZF | | GFRa | | SALL4 | | OCT4 | | CD9 | | Int | | VASA | | KIT | |
| # | (Y) | Diag | Be/Af | IF | R | IF | R | IF | R | IF | R | IF | R | IF | R | IF | R | IF | R |
| 2 | 6 | ALL | Be | + | | − | | − | | + | | + | | + | | + | | + | |
| | | | Af (11 | − | − | − | − | − | − | − | − | − | − | − | − | − | − | − | − |
| | | | Ws) | | | | | | | | | | | | | | | | |
| 3 | 7 | ALL | Be | + | + | − | − | − | + | + | + | + | − | + | − | + | + | + | + |
| | | | Af (15 | − | | + | | − | | + | | − | | + | | + | | − | |
| | | | Ws) | | | | | | | | | | | | | | | | |
| 4 | 10 | ALL | Be | − | + | − | − | − | − | − | − | − | − | − | − | − | − | − | − |
| | | | Af (8 | − | | − | | − | | − | | − | | − | | − | | − | |
| | | | Ws) | | | | | | | | | | | | | | | | |
| 5 | 13 | ALL | Be | | | | + | | | | | | − | | | | | | − |
| | | | Af (8 | | | | + | | | | | | − | | | | | | + |
| | | | Ws) | | | | | | | | | | | | | | | | |
| 6 | 6 | MD | Be | + | | + | + | | | − | | + | + | + | | + | | + | − |
| | | | Af (5 | | | + | − | | | | | + | + | | | | | − | − |
| | | | Ws) | | | | | | | | | | | | | | | | |
| 8 | 7 | THA | Be | + | − | − | − | − | − | + | − | − | − | − | − | + | − | − | − |
| | | | Af (9 | + | | − | | + | | + | | + | | + | | + | | − | |
| | | | Ws) | | | | | | | | | | | | | | | | |

| | | | | | | | | | | Postmeiotic | | | | | |
|---|---|---|---|---|---|---|---|---|---|---|---|---|---|---|---|
| Patients | | | | Meiotie markers | | | | | | markers | | | | | |
| | Age | | Culture | CREM | | LDH | | BOUL | | PRO | | ACR | | ES | Col |
| # | (Y) | Diag | Be/Af | IF | R | IF | R | IF | R | IF | R | IF | R | MT | Size |
| 2 | 6 | ALL | Be | − | | − | | − | | − | | − | | | |
| | | | Af (11 | − | − | − | − | − | − | − | − | − | − | − | S |
| | | | Ws) | | | | | | | | | | | | |
| 3 | 7 | ALL | Be | − | − | − | − | − | + | − | + | − | − | − | |
| | | | Af (15 | − | | | | + | | − | | + | | + | S, M, L |
| | | | Ws) | | | | | | | | | | | | |
| 4 | 10 | ALL | Be | − | + | − | − | − | + | − | − | − | − | | |
| | | | Af (8 | − | | − | | − | | − | | − | | − | S |
| | | | Ws) | | | | | | | | | | | | |
| 5 | 13 | ALL | Be | | − | | + | | | | − | | − | | |
| | | | Af (8 | | − | | − | | | | − | | − | − | S |
| | | | Ws) | | | | | | | | | | | | |
| 6 | 6 | MD | Be | − | − | | − | | | − | − | − | − | | |
| | | | Af (5 | + | + | + | + | | | − | + | − | − | − | S, M |
| | | | Ws) | | | | | | | | | | | | |
| 8 | 7 | THA | Be | − | − | + | − | − | − | − | − | − | − | | |
| | | | Af (9 | − | | − | | + | | − | | − | | − | S, M, L |
| | | | Ws) | | | | | | | | | | | | |

Table 4. “Be” = before culturing; “Af ' = after culturing in the methylcellulose culture system. “Ws” = weeks of culturing. “S” = small; “M” = medium′ “L” = large; “Col” - colonies. “a-6-Int” = alpha 6 integrin. “GFRa” = GFRalpha. “Diag” = diagnosis.

Immunofluorescence Staining and Expression of Spermatogenic Markers in Isolated Cells from Testicular Biopsies of Pre-Pubertal Cancer Patient Boys—

Cells isolated from testicular biopsies of pre-pubertal cancer patient boys (n=8) before and/or after culture in MCS showed positive immuno-fluorescence (IF) staining for cells belonging to the premeiotic stages of spermatogenesis (positive cells were very few, less than 10 cells from a total of around 30 cells. The same scenario existed for meiotic and postmeiotic cells, with a different percentage. It depended on the examined marker and patient, or expression of those markers when examined by RT-PCR analysis (positive expression of markers of the different stages of spermatogenesis is presented in FIG. 4N) (summary of results in Tables 2 and 3). This is not a background IF staining because it was only considered positive staining when it was clearly stained in comparison to the negative control. Representative positive staining of cells from the different stages of spermatogenesis is shown in FIGS. 4A-M and in Tables 2, 3 and 4 above. In addition, expression of spermatogenic markers by PCR analysis is shown in FIG. 4N and Tables 2, 3 and 4 above by positive expression of markers of the different stages of spermatogenesis.

Isolated cells from patient number 4 expressed only plzf, crem-1 and boule, and isolated cells from patient number 8 did not express any spermatogenic marker (Table 2 above).

These results show that testicular tissues from prepubertal patient boys previously exposed to chemotherapy (including cyclophosphamide) contained premeiotic cells that stained for some markers (ranging from 1-7 markers) specific for spermatogonial cells. It should be noted that the two patients (Nos. 4 and 5) at ages 10 and 13 years old, respectively showed staining for only one premeiotic marker (Table 3).

Induction of Spermatogenesis Including the Generation of Sperm-Like Cells in MCS—

In two patients (number 1, number 7) the number of isolated cells from testicular biopsies was very low (about 2×10⁴ cells) and therefore in vitro culture was not performed. Isolated cells from the remaining six testicular biopsies (6 patients) were cultured in MCS for a period of 5-15 weeks (as schematically described in FIG. 1A). During this period cells were developed to form only scattered cells in MCS (single cells, not clusters/colonies) and/or small colonies/clusters (S, up to 30 cells) and/or medium colonies/clusters (M, up to 100 cells) and/or large colonies/clusters (L, >150 cells) (FIGS. 3A-D). Cells/colonies were collected and examined for the different stages of spermatogenesis by IF and/or PCR analyses as shown in Table 4 below. In patient No. 2, colonies started to develop after 8 weeks. Patient No. 3 developed large colonies after 8 weeks of culture. Patient No. 4 developed colonies after 7 weeks of culture. In patient No. 5, the colonies developed after 1 week of culture. In patient No. 6, colonies developed after 2 weeks of culture. Patient No. 8 developed colonies after 1 week of culture. In one biopsy (Patient number 4) the isolated cells expressed before culture only plzf, crem-1 and boule, and in another biopsy (Patient number 2) the isolated cells did not stain any spermatogenic marker (meiotic or post meiotic marker) before culture (but the tissue before cell isolation stained for different premeiotic markers). Cells from both biopsies did not express any spermatogenic marker after in vitro culture in MCS (Tables 2, 3 and 4). As is further shown in Table 4 above, one biopsy expressed some premeiotic markers before and after culture in MCS and developed meiotic (CREM-1, LDH) and postmeiotic (protamine) markers after in vitro culture in MCS. Cells from biopsy number 8 expressed and developed premeiotic markers before and after culture in MCS and also developed only the meiotic marker boule but did not develop postmeiotic markers. However, cells from biopsy of patient number 3 expressed and developed premeiotic markers before and after culture in MCS and also developed meiotic (boule) and postmeiotic (acrosin) markers (Table 4) and even developed sperm-like cells in MCS (FIGS. 3M-3T).

In addition, the present inventor shows that SALL4- and PLZF-positive spermatogonial cells from the developed cultures were co-stained with Ki67 (a marker of proliferation) by double immunostaining (FIGS. 3E-3L), which indicates proliferation of spermatogonial cells in the cultures. Cells/colonies were collected and examined for the different stages of spermatogenesis (FIG. 1C) by IF and/or RT-PCR analyses (FIGS. 4A-M and FIG. 4N, respectively; representative results) (summary of results in Table 3). In general, the number of examined markers was dependent on the amount of RNA extracted from the samples. The present inventor tried to compare markers, which were positive before culture to after culture (for example, BOUL and LDH in patients Nos. 3, 4, 5, and 8). In one biopsy (Patient No. 4), the isolated cells only expressed PLZF, CREM-1 and BOULE before culture, and in another biopsy (Patient No. 2), the isolated cells did not stain for any of the examined spermatogenic markers before culture but stained in the tissue (before cell isolation) for PLZF, OCT4, CD9, α-6-Int., VASA, c-KIT (Table 3). Those cells from both biopsies did not express spermatogenic markers after in vitro culture in MCS (Table 3). Another biopsy (Patient No. 5) expressed some premeiotic markers before and after culture in MCS but did not develop meiotic or postmeiotic markers after culture in MCS (Table 2). These results show that one biopsy (Patient No. 6) expressed 6 premeiotic markers before culture, and after in vitro culture in MCS, it developed meiotic (CREM-1, LDH) and postmeiotic (expression of protamine) cells/markers. Cells from the biopsy of patient No. 8 (THA) expressed and developed premeiotic markers before and after culture in MCS and also developed the meiotic marker BOULE but did not develop postmeiotic markers. Cells from biopsy of patient #3 expressed and developed premeiotic markers before and after culture in MCS and also developed the meiotic marker BOULE (IF) (Table 3) and the postmeiotic marker acrosin in round spermatid (FIGS. 3M and 3N) and elongated-like cells (FIG. 30) (Table 3). Acrosin-stained cells were characterized as round or elongated spermatid, according to their morphology [size and shape of the nucleus; FIG. 3M-O]. In addition, cells with sperm-like morphology that were developed in vitro were identified according to staining with MitoTracker [FIGS. 3Q-S]. In FIG. 3Q, the cytoplasm of sperm-like cells was large (similar to the morphology of i8 and i9 that are characterized in stages 8-12 of spermatid development in the human seminiferous epithelium VIII-XII stages [Muciaccia B, et al. 2013. Biol Reprod. 89:1-10) and stained with MitoTracker (mitochondria in the cytoplasm stained green), while the nucleus with morphology of sperm-like cell nucleus is stained blue. It should be noted that some sperm-like cells showed a nucleus similar in their size [FIG. 30 and FIG. 3Q] to normal sperm found in some biopsies of non-obstructive azoospermic patients [FIG. 3T] even though the final morphological structure was different. However, other cells with sperm-like morphology exhibited a relatively large nucleus [FIGS. 3R and 3S] compared to the control [FIG. 3T] but presented a neck (N) (with concentrated green color which indicates the presence of concentrated mitochondria) and tail. These morphologies of sperm-like cells that developed in MCS are similar to those morphologies characterized in stages 8-12 of spermatid development in the human seminiferous epithelium VIII-XII stages [Muciaccia B, et al. 2013. Biol Reprod. 89:1-10].

In summary, following culture in MCS, premeiotic markers were developed in at least 3/6 biopsies, meiotic markers were developed in 3/6 biopsies and post meiotic markers were developed in 2/6 biopsies. Cells with sperm-like morphology were identified in 1/6 of the biopsies (FIGS. 3M-T, Table 4).

### Example 2

### Maturation of Sperm Using Adult Spermatogonia

Experimental Results

Germ Cells from Testicular Biopsies of Different Azoospermic Patients could Form Single Cells and/or Small, Medium and Large Colonies in MCS Culture—

The development of cells/colonies from biopsies without (−) or with (+) sperm of the different groups of azoospermic patients were examined for several weeks/months in MCS. The development of cells in MCS was divided according to: cells (single, pair, aline, and colonies (small, medium and large). The results are summarized in Table 5 below.

TABLE 5
Summary of developed cell/colonies in vitro from isolated
cells of testicular biopsies without or with sperm

| Diag./colonies | | | | | |
|---|---|---|---|---|---|
| develop. | Sperm | Cells | Small | Medium | Large |
| All | − | 2/17 (12%) | 9/17 (53%) | 4/17 (24%) | 2/17 (12%) |
| | + | 8/20 (40%) | 7/20 (35%) | 5/20 (25%) | 0/20 (0%) |

Table 5: The azoospermic patients (marked “All”, n = 37) were divided according to the findings of sperm in IVF lab into azoospermic patients with (the “sperm” column is marked as “+”; n = 20) and without sperms (the “sperm” column is marked as “−”; n = 17).

Developed colonies/cells from a biopsy without sperms of a Sertoli cell only syndrome azoospermic patient were examined for RNA expression of spermatogenic markers by PCR analysis using specific primers and/or by immunofluorescence staining using specific antibodies for each of the spermatogenic markers: pre-meiotic (OCT-4, PLZF, VASA, GFRA1, CD-9, a-6-INTEGRIN, SALL4 and c-KIT), meiotic (CREM1, LDH and BOULE) and post-meiotic (protamine and acrosin). The expression/presence of the spermatogenic markers was examined during 4-16 weeks in culture compared to the expression before culture. The category of azoospermic patients without sperms was defined according to the finding in IVF lab.

TABLE 6
Expression of spermatogenic markers in colonies/cells developed
in vitro from a biopsy without sperm of an azoospermic patient

| Post- | | | | | | | | | | | | | Stage | | |
|---|---|---|---|---|---|---|---|---|---|---|---|---|---|---|---|
| miotic | | Miotic | | | Pre-miotic | | | | | | | | Marker | | |
| acr | prot | boule | ldh | crem | ckit | sall4 | a-6 | cd9 | gfra | vasa | plzf | oct4 | weeks | | BC/AC |
| + | − | − | − | − | − | + | − | + | + | + | + | + | BC | | |
| − | − | − | + | − | − | + | − | − | − | − | − | + | 5 | w | AC |
| − | − | − | + | − | − | + | − | + | + | − | − | + | 9 | w | |
| − | − | − | + | − | − | + | − | + | + | − | − | + | 9 | w | |
| − | − | − | + | − | − | + | − | − | − | − | − | + | 11 | w | |
| − | − | − | − | − | − | + | − | − | − | − | − | − | 12 | w | |
| + | + | + | + | − | − | + | − | + | + | + | − | + | 13 | w | |
| − | − | − | − | − | − | − | − | − | − | − | − | − | 14 | w | |
| − | − | − | − | − | − | − | − | − | − | − | − | − | 16 | w | |

Table 6. “BC” = before culture; “AC” = after in vitro culture in MCS, “w” = weeks. Red color indicates changes in the expression of meiotic or postmeiotic markers during culture in MCS.

Developed colonies/cells from testicular biopsies with and without sperms of azoospermic patients were examined for the presence of spermatogenic markers by PCR analysis using specific primers and/or by immunofluorescence staining using specific antibodies for each of the markers: pre-meiotic (OCT-4, PLZF, VASA, GFRA1, CD-9, A-6-INTEGRIN, SALL4 and c-KIT), meiotic (crem1, ldh and boule) and post-meiotic (protamine and acrosin). The category of azoospermic patients with sperms was defined according to the finding of individual/few sperms in IVF lab.

TABLE 8
Expression of spermatogenic markers in colonies/cells developed
in vitro from biopsies without sperm of azoospermic patients
Stage

| | | | | | | | | | | | Post- | |
|---|---|---|---|---|---|---|---|---|---|---|---|---|
| Pre-meiotic | | | | | | | | Meiotic | | | meiotie | |
| oct4 | plzf | vasa | gfr | cd9 | a-6 | sall4 | ckit | crem1 | ldh | boule | prot | acr |
| 0/7 | 3/7 | 3/7 | 1/7 | 3/7 | 2/7 | 0/7 | 0/7 | 0/7 | 2/7 | 2/7 | 1/7 | 1/7 |
| 0% | 43% | 43% | 14% | 43% | 28% | 0% | 0% | 0% | 28% | 28% | 14% | 14% |

Table 8. Before culture all of the pre-meiotic, meiotic and post-meiotic markers were negative; Shown are the frequencies of the samples which express the indicated markers after in vitro culturing in MCS).

TABLE 9
Expression of spermatogenic markers in colonies/cells developed
in vitro from biopsies with sperm, of azoospermic patients
Stage

| | | | | | | | | | | | Post- | |
|---|---|---|---|---|---|---|---|---|---|---|---|---|
| Pre-meiotic | | | | | | | | Meiotic | | | meiotic | |
| oct4 | plzf | vasa | gfr | cd9 | a-6 | sall4 | ckit | crem1 | ldh | boule | prot | acr |
| 4/14 | 4/14 | 7/14 | 6/14 | 1/14 | 2/14 | 4/14 | 1/14 | 3/14 | 2/14 | 3/14 | 4/14 | 7/14 |
| 28% | 28% | 50% | 43% | 7% | 14% | 28% | 7% | 21% | 14% | 21% | 28% | 50% |

Table 9: Before culture all of the pre-meiotic, meiotic and post-meiotic markers were negative; Shown are the frequencies of samples which express the indicated markers after in vitro culturing in MCS.

Note the remarkable decrease in the PLZF, CD9, A-6-INTEGRIN, and LDH markers between biopsies without (Table 6) and with sperm (Table 7). On the other hand, note the remarkable increase in the GFR, SALL4, CREM1, PROT, and ACR examined markers between biopsies without (Table 6) and with sperm (Table 9).

### Example 3

### Differentiation of Spermatogonial Stem Cells in a Medium Comprising TNF-Alpha (TNFα)

Isolated testicular cells (from azoospermic adult patients) were cultured in methylcellulose culture system (MCS) in medium alone (stemPro and growth factors as described in the “general materials and experimental methods”) (control; CT), or with the addition of tumor necrosis alpha (TNFα; 20 pg/ml) for 14.5 weeks (Ws) for patient (No. 1). It is noted that TNFα can be used at a concentration range of 1-200 pg/ml.

The expression of the different spermatogenic markers were examined by immunofluorescence staining using specific antibodies for each marker before culture (BC) and after culture (AC) in methylcellulose culture system (MCS) after 14.4 weeks. The results are presented in Table 10 below.

TABLE 10
Effect of TNFα on development of spermatogenic markers in vitro (MCS)

| | | | Spermatogenesis markers | | | | | |
|---|---|---|---|---|---|---|---|---|
| | | | | | | | | Post- |
| Patient | | | Pre-meiotic | | | Meiotic | | meiotic |
| # | Treatment | BC/AC | VASA | PLZF | SALL4 | CREM | BOULE | ACROSIN |
| 1 | | BC | + | + | + | − | + | + |
| | CT | AC | + | + | + | − | + | + |
| | TNFα | (14.5 Ws) | + | + | − | + | + | + |

Table 10: “+” indicates positive staining for the indicated marker, “−” indicates negative staining for the indicated marker. “BC” = before culture: “AC” = after culture; “Ws.” = weeks.

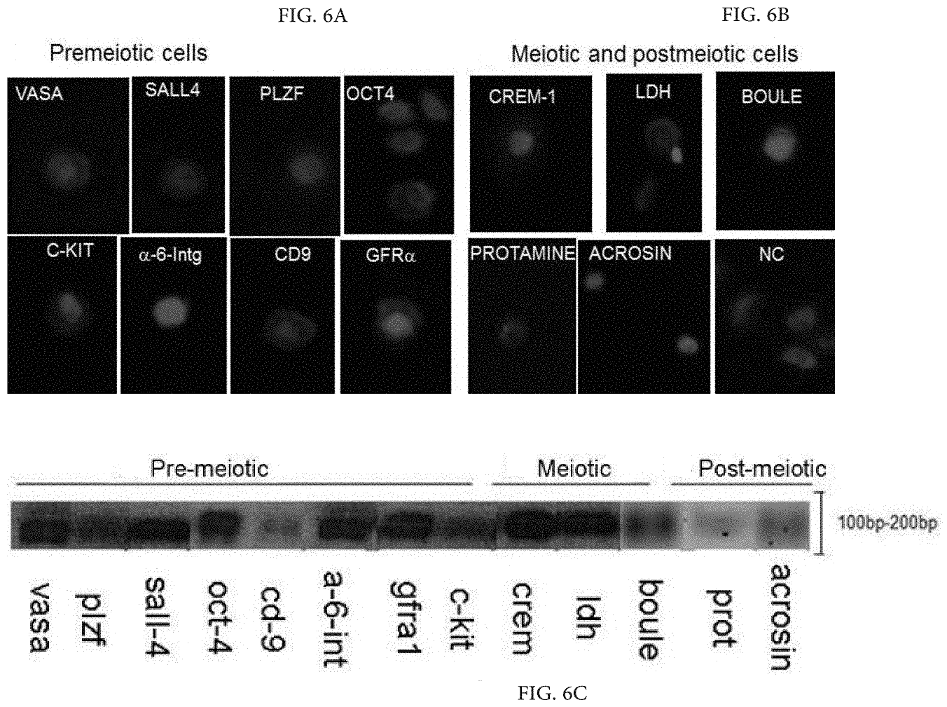

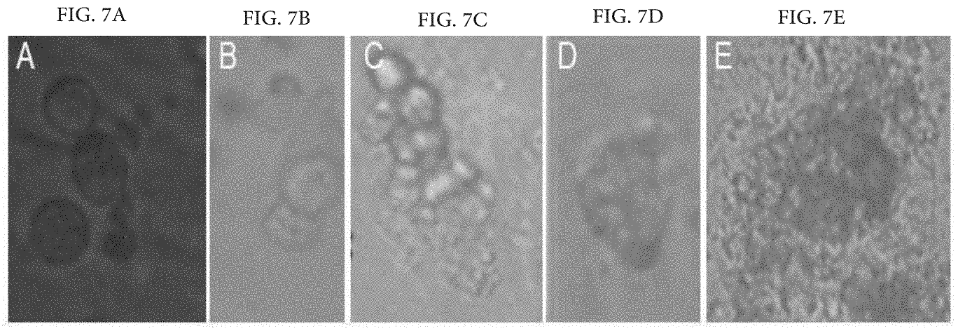

These results show that colonies were developed in MCS in the presence or absence of TNFα (FIGS. 7A-B). However, the presence of TNFα in culture of patient #1 maintained/induced growth of premeiotic cells VASA and PLZF, and the meiotic cells (Boule) and the postmeiotic cells (acrosin positive cells) in a similar manner as control (CT) (Table 8). However, TNFα induced the differentiation of the pre-meiotic cells to meiotic cells that express CREM compared to CT (did not induce CREM) (Table 10).

It should be noted that “CREM” (Gene ID: 1390), a cAMP responsive element modulator, is a bZIP transcription factor that binds to the cAMP responsive element found in many viral and cellular promoters. It is an important component of cAMP-mediated signal transduction during the spermatogenetic cycle, as well as other complex processes. It should be noted that up until to-date there is no evidence that human spermatogonia can be differentiated in vitro up to the stage of expressing the CREM-1 differentiation marker, using either agar-based culturing system or methylcellulose based culturing system.

Without being bound by any theory, these results may indicate possible involvement of TNFα in the development of human meiotic cells in vitro.

### Example 4

### Effect of Hormones and Retinoic Acid on the Development of Mouse Spermatogenesis In Vitro in Methylcellulose Culture System

In the following example, the present inventor tested the effect of the testosterone (T) and the follicle stimulating hormone (FSH) hormones as well as of retinoic acid (RA) on the development of mouse spermatogenesis in vitro, using the methylcellulose culture system.

Cells were enzymatically isolated from seminiferous tubules of ICR mice (which were developed by the Institute of Cancer Research, also called CD-1) immature mice (7-day-old mice). These cells were cultured in vitro in methylcellulose culture system essentially as described in the “General Materials and Experimental Methods” section above, with some modifications to the culture medium as described below.

Briefly, cells were cultured (2×10⁵ cells/well/500 μl) in MEM medium which contained sodium bicarbonate 7.5%, L-glutamine 200 mM, non-essential amino acids 1%, penicillin/streptomycin and gentamicin 10 mg/ml, and were incubated over 2 nights in 24 wells plate at 37° C., 5% CO2. The nonadherent cells were collected and cultured (2×10′ cells/well/500 μl) in methylcellulose (42%; R&D, Minneapolis, USA) [as a three-dimension (3D) culture system], which contained the StemPro-34 medium (Gibco, USA) enriched with: 10% KSR (knockout serum replacement, Gibco, USA) and the following growth factors: Glial cell-derived neurotrophic factor (GDNF; 10 ng/ml; Biolegend), Leukemia inhibitory factor (LIF; 10 ng/ml; Biolegend), epidermal growth factors (EGF; 20 ng/ml; Biolegend, CA, USA), bFGF (basic fibroblast growth factor) (10 ng/ml; Biolegend).

Cells were cultured for 4 weeks. Every 1-2 weeks (according to the growth and morphology of the cells) the present inventor added 50 μl/well of fresh concentrated (×10) enriched StemPro-34 medium to the cell cultures.

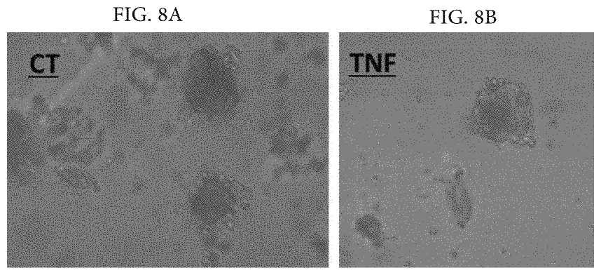

After two weeks of culture, various concentrations of testosterone (T) such as 10⁻⁸ M, 10⁻⁷ M or 10⁻⁶ M (as shown in FIG. 8A), or retinoic acid (“RA”, such as 10⁻⁸ M, 10⁻⁷ M or 10⁻⁶ M as shown in FIG. 8C) were added to the cultures. After additional two weeks of culture (total culture of four weeks) cells were collected from the in vitro culture system, and examined for different markers of spermatogenesis: pre-meiotic markers such as: VASA, CD9, meiotic markers such as: CREM and BOULE and post-meiotic markers such as ACROSIN by qPCR analysis.

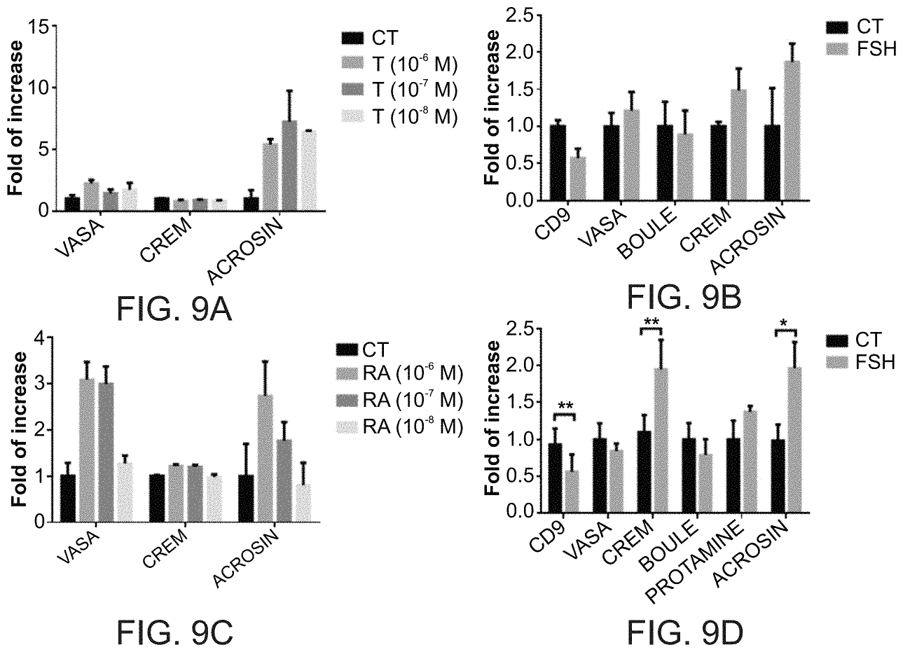

As shown in FIGS. 9A and 9C, the addition of testosterone (T) increased mainly the expression of the meiotic/post-meiotic marker ACROSIN (FIG. 9A). The addition of retinoic acid (RA) increased mainly the expression of the pre-meiotic marker VASA and the meiotic/post-meiotic marker ACROSIN (FIG. 9C).

Testing the Effect of FSH—

In other cultures, follicle stimulating hormone (FSH) was added at a final concentration of 25 U/ml FSH to isolated cells from normal mice. As is shown in FIG. 9B the addition of FSH increased the expression of the meiotic marker CREM and the meiotic/post-meiotic marker ACROSIN (FIG. 9B).

Mimicking Human Azoospermic Syndrome by a Chemotherapy Drug—

In order to mimic the human azoospermic syndrome (no development of sperm in the testes), the present inventor has treated immature (7-day-old) ICR mice with busulfan (BU) (45 mg/kg) (a chemotherapy agent). After 10 days of the treatment, testes were removed and cells from the seminiferous tubules were enzymatically isolated as mentioned above for normal (untreated) mice. The isolated cells were cultured as mentioned above with the StemPro medium supplemented with KSR and the following growth factors: GDNF (10 ng/ml), LIF (10 ng/ml), EGF (20 ng/ml) and bFGF (10 ng/ml), as well as with FSH (25 U/ml FSH) from the beginning of the culture. After four weeks of culture, cells were collected and examined for the development of spermatogenesis in vitro as mentioned above.

As is shown in FIG. 9D the addition of FSH significantly increased the expression of the meiotic marker CREM and the meiotic/post-meiotic marker ACROSIN (FIG. 9D).

Thus, these results show that addition of testosterone, FSH or retinoic acid in vitro to spermatogonial cells from normal immature mice or immature mice treated with busulfan (BU) increased the expression of meiotic/post-meiotic markers.

In conclusion, addition of testosterone, FSH and retinoic acid may induce development of spermatogenesis in vitro.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Additional References are Cited in Text

1. Aslam I, Fishel S, Moore H, Dowell K, Thornton S. Fertility preservation of boys undergoing anti-cancer therapy: a review of the existing situation and prospects for the future. Hum Reprod. 2000 October; 15(10):2154-9.
2. Desandes E. Survival from adolescent cancer. Cancer Treat Rev 2007; 33:609-615.
3. Hudson M M. Reproductive outcomes for survivors of childhood cancer. Obstet Gynecol 2010; 116:1171-1183.
4. Ward E, De Santis C, Robbins A, Kohler B, Jemal A. Childhood and adolescent cancer statistics, 2014. CA Cancer J Clin 2014; 64:83-103.
5. Kliesch S, Behre H M, Jürgens H, Nieschlag E. Cryopreservation of semen from adolescent patients with malignancies. Med Pediatr Oncol. 1996 January; 26(l):20-7.
6. Gassei K, Orwig K E. Experimental methods to preserve male fertility and treat male factor infertility. Fertil Steril. 2016 February; 105(2):256-66. doi: 10.1016/j.fertnstert.2015.12.020. Epub 2015 Dec. 30.
7. Picton H M, Wyns C, Anderson R A, Goossens E, Jahnukainen K, Kliesch S, Mitchell R T, Pennings G, Rives N, Tournaye H, van Pelt A M, Eichenlaub-Ritter U, Schlatt S. A European perspective on testicular tissue cryopreservation for fertility preservation in prepubertal and adolescent boys. Hum Reprod. 2015 November; 30(11):2463-75. doi: 10.1093/humrep/dev190. Epub 2015 Sep. 10.
8. Mahmoud H. Spermatogenesis in an artificial three-dimensional system. Stem Cells. 2012 November; 30(11):2355-60. doi: 10.1002/stem.1238.
9. Jahnukainen K, Stukenborg J B. Present and future prospects of male fertility preservation for children and adolescents. J Clin Endocrinol Metab. 2012 December; 97(12):4341-51. doi: 10.1210/jc.2012-3065. Epub 2012 Oct. 4.
10. Fujita K, Tsujimura A, Miyagawa Y, Kiuchi H, Matsuoka Y, Takao T, et al. Isolation of germ cells from leukemia and lymphoma cells in a human in vitro model: potential clinical application for restoring human fertility after anticancer therapy. Cancer Res 2006; 66:11166-71.
11. Geens M, Goossens E, Tournaye H. Cell selection by selective matrix adhesion is not sufficiently efficient for complete malignant cell depletion from contaminated human testicular cell suspensions. Fertil Steril 2011; 787:91-95.
12. Dovey S L, Valli H, Hermann B P, Sukhwani M, Donohue J, Castro C A, et al. Eliminating malignant contamination from therapeutic human spermatogonial stem cells. J Clin Invest 2013; 123:1833-43.
13. Nagano M, Ryu B Y, Brinster C J, Avarbock M R, Brinster R L. Maintenance of mouse male germ line stem cells in vitro. Biol Reprod 2003; 68: 2207-2214.
14. Sadri-Ardekani H, Akhondi M A, van der Veen F, Repping S, van Pelt A M M. In vitro propagation of human prepubertal spermatogonial stem cells. J Am Med Asso 2011; 305: 2416-2418.
15. Sadri-Ardekani H, Mizrak S C, van Daalen S K M, et al. Propagation of human spermatogonial stem cells in vitro. J Am Med Asso 2009; 19:2127-2134.
16. Sato T, Katagiri K, Gohbara A, Inoue K, Ogonuki N, Ogura A, Kubota Y, Ogawa T. In vitro production of functional sperm in cultured neonatal mouse testes. Nature 2011; 471:504-507.
17. Abu Elhija M, Lunenfeld E, Schlatt S, Huleihel M. Differentiation of murine male germ cells to spermatozoa in a soft agar culture system. Asian J Androl 2012; 14:285-293.
18. Stukenborg J-B, Schlatt S, Simoni M, Yeung C-H, Abu Elhija M, Luetjens C M, Huleihel M, Wistuba J. New horizons for in vitro spermatogenesis? An update on novel three-dimensional culture systems as tools for meiotic and post-meiotic differentiation of testicular germ cells. Mol Hum Reprod 2009; 15: 521-529.
19. Stukenborg J B, Wistuba J, Luetjens C M, Elhija M A, Huleihel M, Lunenfeld E, Gromoll J, Nieschlag E, Schlatt S. Coculture of spermatogonia with somatic cells in a novel three-dimensional soft-agar-culture-system. J Androl. 2008 May-June; 29(3):312-29. Epub 2007 Nov. 28.
20. Huleihel M, Nourashrafeddin S, Plant™. Application of three-dimensional culture systems to study mammalian spermatogenesis, with an emphasis on the rhesus monkey (Macaca mulatta). Asian J Androl 2015; 17:972-980.
21. Hermann B P, Sukhwani M, Hansel M C, Orwig K E. Spermatogonial stem cells in higher primates: are there differences from those in rodents? Reproduction 2010; 139:479-493.
22. Kanatsu-Shinohara M, Ogonuki N, InoueK, et al., “Long-term proliferation in culture and germline transmission of mouse male germline stem cells,” Biol Reprod 2003; 69: 612-616.
23. Ginsberg J P, C A Carlson, K Lin, W L Hobbie, E Wigo, X Wu, R L Brinster, T F Kolon. (2010). An experimental protocol for fertility preservation in prepubertal boys recently diagnosed with cancer: a report of acceptability and safety. Hum Reprod 25: 37-41.
24. Wyns C, M Curaba, S Petit, B Vanabelle, P Laurent, J-F X Wese, J Donnez. (2011). Management of fertility preservation in prepubertal patients: 5 years' experience at the Catholic University of Louvain. Hum Reprod 26: 337-347.
25. Wyns C, C Collienne, F Shenfield, A Robert, P Laurent, L Roegiers, B Brichard. (2015). Fertility preservation in the male pediatric population: factors influencing the decision of parents and children. Hum Reprod 30: 2022-2030.
26. Uijldert M, A Meilner, A A de Melker, AMM van Pelt, M D van de Wetering, R R van Rijn, M van Wely, F van der Veen, S Repping. (2017). Development of the testis in pre-pubertal boys with cancer after biopsy for fertility preservation. Hum Reprod 32: 2366-2372.
27. Jahnukainen K, M Hou, C Petersen, B Setchell, O So\ddot{ }der. (2001). Intratesticular transplantation of testicular cells from leukemic rats causes transmission of leukemia. Cancer Res 61: 706-710.
28. Mulder C L, L A E Catsburg, Y Zheng, C M deWinter-Korver, vanDaalen, S K M, M vanWely, S Pals, S Repping, A M M vanPelt. (2018). Long-term health in recipients of transplanted in vitro propagated spermatogonial stem cells. Hum Reprod 33: 81-90.
29. Wu X, J A Schmidt, M R Avarbock, J W Tobias, C A Carlson, T F Kolon, J P Ginsberg, R L Brinster. (2009). Prepubertal human spermatogonia and mouse gonocytes share conserved gene expression of germline stem cell regulatory molecules. Proc. Nat. Acad. Sci 106: 21672-21677.

1. A method of in vitro maturation of human spermatogonium, comprising culturing said spermatogonium in a three-dimensional methylcellulose culture system (MCS) under conditions capable of differentiating said human spermatogonium into an elongated spermatid, thereby in vitro maturing the human spermatogonium.

2. The method of claim 1, wherein said conditions comprise culturing said human spermatogonium in a culture medium which comprises an effective concentration of at least one growth factor selected from the group consisting of Glial cell line-derived neurotrophic factor (GDNF), leukemia inhibitory factor (LIF), basic fibroblast growth factor (bFGF), and epidermal growth factor (EGF).

3. The method of claim 2, wherein said culture medium further comprises TNFalpha (TNFα).

4. The method of claim 2, wherein said culture medium further comprises at least one agent selected from the group consisting of: testosterone, follicle stimulating hormone (FSH) and retinoic acid.

5. The method of claim 2, wherein culture medium comprises serum replacement.

6. The method of claim 1, wherein said culture medium comprises STEM PRO® (Thermo Fisher Scientific) supplement.

7. The method of claim 2, wherein said culture medium further comprises at least one hormone selected from the group consisting of: Follicle-Stimulating Hormone (FSH) and testosterone.

8. The method of claim 7, wherein culturing in the presence of said at least one hormone is performed following about one month of culturing in the presence of said at least one growth factor.

9. The method of claim 7, wherein said at least one hormone is added to said culture medium which comprises said at least one growth factor.

10. The method of claim 1, wherein said human spermatogonium is comprised in a testicular biopsy of the subject.

11. The method of claim 10, wherein said testicular biopsy is obtained from a prepubertal male subject.

12. The method of claim 10, wherein said testicular biopsy is obtained from a non-obstructive azoospermic patient.

13. The method of claim 1, further comprises identifying a meiotic cell, a post meiotic cell and/or a mature sperm cell following said culturing in vitro.

14. The method of claim 1, further comprises identifying a cell expressing CREM (cAMP responsive element modulator), following said culturing in vitro.

15. An in vitro matured sperm obtainable according to the method of claim 1.

16. A cell obtainable according to the method of claim 1, wherein said cell is characterized by at least the expression of CREM (cAMP responsive element modulator).

17. A method of treating a subject in need of mature sperm cells, comprising:
(a) obtaining a spermatogonium from the subject, and
(b) subjecting said spermatogonium to an in vitro maturation according to the method of claim 1,
thereby generating mature sperm cells of the subject, and treating the subject.

18. The method of claim 17, wherein the subject is a prepubertal male subject.

19-25. (canceled)

26. The method of claim 17, wherein the subject is a non-obstructive azoospermic patient.

27. The method of claim 26, wherein said subject is in need of aggressive chemotherapy and/or aggressive radiotherapy.
